(19) United States
(12) Patent Application Publication
(10) Pub. No.: US 2017/0112857 A1
(43) Pub. Date: Apr. 27, 2017

(54) Extended Release Aspirin

(71) Applicant: New Haven Pharmaceuticals, Inc., North Haven, CT (US)

(72) Inventors: Larry Dillaha, Paradise Valley, AZ (US); Jeff Patrick, Chesterfield, MO (US)

(21) Appl. No.: 15/131,446

(22) Filed: Apr. 18, 2016

Related U.S. Application Data
(60) Provisional application No. 62/246,659, filed on Oct. 27, 2015.
(60) Provisional application No. 62/246,944, filed on Oct. 27, 2015.

(51) Int. Cl. A61K 31/616; A61K 9/00

(52) U.S. Cl. CPC A61K 31/616; A61K 9/0053

(57) ABSTRACT

The present invention is directed to methods of inhibiting platelet aggregation, reducing serum thromboxane B2 levels, reducing systemic or cardiovascular inflammation, treating or preventing cancer and treating or preventing cardiovascular disease by oral administration of compositions containing extended release acetylsalicylic acid (ASA) or a combination of extended release ASA and immediate release ASA.

### FIELD OF THE INVENTION

The present invention is directed to extended release aspirin compositions and methods of their use. The present invention is further directed to the treatment and prevention of systemic or cardiovascular inflammation, cardiovascular disease, cancer and diseases associated with increased thrombotic risk factors due to platelet activation, aggregation or production. The present invention is further directed to inhibition of platelet aggregation.

### BACKGROUND OF THE INVENTION

Cardiovascular diseases (i.e. heart disease) are diseases of the heart and blood vessels. Heart disease is the number one cause of death in both men and women in the United States. Specifically, about 610,000 people die from heart disease each year. Heart disease is generally the result of narrowed or blocked blood vessels. Blood vessels become narrowed or blocked through a process known as atherosclerosis. Atherosclerosis is the build-up of plaque in blood vessels. This plaque may tear away from the blood vessel causing a rupture which is quickly filled with platelets. These platelets aggregate at the site of the rupture and often times completely block blood flow within that blood vessel leading to angina, heart attacks or stroke.

Platelet aggregation also plays an important role in cancer and cancer progression. The growth of a tumor relies on new blood vessels (i.e. angiogenesis) to supply the growing tumor with oxygen and other nutrients. Activated platelets release a multitude of proangiogenic factors including one of the most potent, vascular endothelial growth factor (“VEGF”.) In fact, activated platelets are the main transporter of VEGF. Verheul H M et al., Platelet: transporter of vascular endothelial growth factor, Clin Cancer Res, 1997 December, 3(12 Pt 1), 2187-2190. Further, activated platelets shed platelet-derived microparticles (“PMP”.) Studies have shown that PMP's stimulate both proliferation and adhesion of cancer cells. Varon D et al., Role of platelet-derived microparticles in angiogenesis and tumor progression, Discov Med, 2009 December, 8(43), 237-241. However, to become activated, platelets must attach to the extra-cellular matrix and subsequently aggregate. Sabrkhany S et al., The role of blood platelets in tumor angiogenesis, Biochim Biophys Acta, 2011 April, 1815(2). 189-196. Further, inhibition of platelet aggregation disrupts the interaction of platelets with circulating tumor cells enabling the immune system to attack these cells and thus prevent metastasis. Sinha G, More evidence that aspirin lowers cancer risk, J Natl Cancer Inst, 2015 Jan. 6, 107(1), 495. Acetylsalicylic acid (“ASA”) (or aspirin) inhibits platelet aggregation by inhibiting the conversion of arachidonic acid to thromboxane by cyclooxygenase (“COX”) enzymes in the liver. Inhibiting thromboxane production is a key aspect of inhibiting platelet aggregation because thromboxane is a platelet aggregation stimulator. In the absence of a COX inhibitor thromboxane production is rapid and copious. Additionally, platelet production occurs around the clock, including substantial periods of time when ASA has been cleared from the blood. This production of “uninhibited platelets” is elevated in diseases such as Diabetes Mellitus and other prothrombotic states. Thus, constant inhibition of the COX enzyme is required for patients in need of platelet aggregation inhibition. ASA inhibits COX enzymes by acetylation, which in turn deacetylates the ASA into salicylic acid (“SA”). The problem with the use of ASA to inhibit platelet aggregation is that excess amounts of ASA pass through the liver deacetylated and enter the vasculature and gastric endothelium. Once in the vasculature and gastric endothelium ASA then acetylates COX enzymes responsible for the production of prostaglandins including prostacyclin. The inhibition of prostacyclin production is counter-productive because prostacyclin itself is a platelet aggregation inhibitor. Thus, maintaining constant but low concentrations of ASA is required for optimal platelet aggregation inhibition.

Overexpression of COX enzymes has also been implicated in a variety of specific cancer types. In breast cancer, overexpression of COX enzymes has been shown to inhibit apoptosis and weaken immune response. Kim et al., Lifetime use of nonsteroidal anti-inflammatory drugs and breast cancer risk: results from a prospective study of women with a sister with breast cancer, BMC Cancer. 2015 Dec. 16, 15, 960. In colorectal cancer, overexpression of COX enzymes is correlated with a lower survival rate among patients. Peng et al., Prognostic significance of COX-2 immunohistochemical expression in colorectal cancer: a meta-analysis of the literature, PLoS One, 2013, 8(3), e58891.

Thrombotic events in patients with essential thrombocytosis (“ET”) being treated with chronic aspirin regiments (100 mg/day) has also been linked to overexpression of COX enzymes. Dragani et al., The contribution of cyclooxygenase-1 and -2 to persistent thromboxane biosynthesis in aspirin-treated essential thrombocythemia: implications for antiplatelet therapy, Blood, 2010 Feb. 4, 115(5), 1054-1061. Dragani et al. hypothesize that the overexpression of COX enzymes is due to faster platelet regeneration in ET patients. Dragani et al. suggests that the 24-hour dosing interval of ASA given to ET patients is sufficient to fully acetylate (i.e. deactive) platelet COX-1 in normal subjects but not in ET patients due to the higher rate of platelet regeneration and thus COX-1 expression. Dragani et al. further suggest that the 24-hour dosing is unsuccessful due to the short half-life of intact ASA in the blood stream.

One way to deliver constant low concentrations of ASA is via a controlled-release ASA formulation. Previously described formulations include those disclosed in U.S. Pat. No. 5,603,957 (“the '957 Patent”), which is directed to microcapsules for the controlled release of ASA. The '957 Patent describes microcapsules containing 160 mg of ASA and a coating material containing 21.5 mg of ethyl cellulose as a film-forming polymer, 2 mg polyvidone as a water soluble polymer, 2 mg castor oil as a plasticizer and 3 mg magnesium stearate as a lubricant, which is then optionally mixed with colloidal silica and talc. These microcapsules are encapsulated in gelatin capsules of 320 mg or 160 mg ASA doses.

However, there is a need in the art for improved methods and compositions for inhibiting platelet aggregation, preventing or treating diseases associated with increased thrombotic risk due to platelet activation, aggregation or production, preventing or treating cardiovascular disease, reducing systemic and cardiovascular inflammation and preventing or treating cancer.

### SUMMARY OF THE INVENTION

In one aspect, the present invention is directed to a method of inhibiting platelet aggregation comprising orally administering to a human in need thereof a controlled-release aspirin composition comprising acetylsalicylic acid (“ASA”) at an amount from about 81 milligrams to about 325 milligrams, preferably from about 162.5 milligrams to 325 milligrams, wherein the method provides a level of platelet aggregation inhibition within about 1 hour of administration and wherein the level of platelet aggregation inhibition remains significantly unchanged from about 1 hour after administration to at least about 24 hours after administration.

In a preferred aspect, the present invention is directed to a method of inhibiting platelet aggregation comprising orally administering to a human in need thereof a controlled-release aspirin composition comprising ASA at an amount of about 325 milligrams, wherein the method provides a level of platelet aggregation inhibition within about 1 hour of administration and wherein the level of platelet aggregation inhibition remains significantly unchanged at least about 24 hours after administration and wherein the level of platelet aggregation inhibition is significantly increased 12 hours after administration as compared to 1 hour after administration.

In another preferred aspect, the present invention a method of inhibiting platelet aggregation comprising orally administering to a human in need thereof a controlled-release aspirin composition comprising ASA at an amount from about 81 milligrams to about 325 milligrams, preferably from about 162.5 milligrams to 325 milligrams, wherein the method provides a level of platelet aggregation inhibition within about 1 hour of administration and wherein the level of platelet aggregation inhibition remains significantly unchanged from about 1 hour after administration to at least about 24 hours after administration and wherein the level of platelet aggregation inhibition is measured by a turbidimetric based optical detection system.

In another preferred aspect, the present invention a method of inhibiting platelet aggregation comprising orally administering to a human in need thereof a controlled-release aspirin composition comprising ASA at an amount of about 325 milligrams, wherein the method provides a level of platelet aggregation inhibition within about 1 hour of administration and wherein the level of platelet aggregation inhibition remains significantly unchanged from about 1 hour after administration to at least about 24 hours after administration and wherein the level of platelet aggregation inhibition is significantly increased 16 hours after administration as compared to 1 hour after administration, and wherein the level of platelet aggregation inhibition is measured by a turbidimetric based optical detection system.

In another aspect, the present invention is directed to a method of reducing serum thromboxane B2 levels comprising orally administering to a human in need thereof a controlled-release aspirin composition comprising ASA at an amount from about 81 milligrams to about 325 milligrams, preferably from about 162.5 milligrams to 325 milligrams, wherein the method provides a reduced serum thromboxane B2 level within 1 hour of administration and wherein the serum thromboxane level remains significantly unchanged from about 1 hour after administration to at least about 24 hours after administration, preferably the method provides no significant change in urinary levels of thromboxane B2.

In another preferred aspect, the present invention is directed to a method of reducing serum thromboxane B2 levels comprising orally administering to a human in need thereof a controlled-release aspirin composition comprising ASA at an amount from about 81 milligrams to about 325 milligrams, preferably from about 162.5 milligrams to 325 milligrams, wherein the method provides a reduced serum thromboxane B2 level within 1 hour of administration and wherein the serum thromboxane level remains significantly unchanged from about 1 hour after administration to at least about 24 hours after administration and wherein the administration of 325 milligrams ASA provides a significantly lower serum thromboxane B2 level as compared to administration of 162.5 milligrams ASA.

In another preferred aspect, the present invention is directed to a method of reducing serum thromboxane B2 levels comprising orally administering to a human in need thereof a controlled-release aspirin composition comprising ASA at an amount of about 81 milligrams, wherein the method provides a reduced serum thromboxane B2 level within 1 hour of administration and wherein the serum thromboxane level remains significantly unchanged from about 1 hour after administration to at least about 24 hours after administration and wherein the method provides no significant change in urinary levels of 2,3-dinor-6-keto-PGF1α, the durable metabolite and surrogate of prostacyclin.

In another preferred aspect, the present invention is directed to a method of reducing serum thromboxane B2 levels comprising orally administering to a human in need thereof a controlled-release aspirin composition comprising ASA at an amount of about 162.5 milligrams, wherein the method provides a reduced serum thromboxane B2 level within 1 hour of administration and wherein the serum thromboxane level remains significantly unchanged from about 1 hour after administration to at least about 24 hours after administration and wherein the method provides lower urinary thromboxane B2 levels than after administration of the controlled-release aspirin composition comprising about 81 milligrams ASA.

In another aspect, the present invention is directed to a method of treating or preventing diseases associated with increased thrombotic risk due to platelet activation, aggregation or production comprising orally administering to a human in need thereof a controlled-release aspirin composition comprising acetylsalicylic acid (“ASA”) at an amount from about 81 milligrams to about 325 milligrams, preferably from about 162.5 milligrams to 325 milligrams, wherein the method provides a level of platelet aggregation inhibition within about 1 hour of administration and wherein the level of platelet aggregation inhibition remains significantly unchanged from about 1 hour after administration to at least about 24 hours after administration.

In another aspect, the present invention is directed to a method of treating or preventing diseases associated with increased thrombotic risk due to platelet activation, aggregation or production comprising orally administering to a human in need thereof a controlled-release aspirin composition comprising acetylsalicylic acid (“ASA”) at an amount from about 81 milligrams to about 325 milligrams, preferably from about 162.5 milligrams to 325 milligrams, wherein the disease is essential thrombocytosis and wherein the method provides a level of platelet aggregation inhibition within about 1 hour of administration and wherein the level of platelet aggregation inhibition remains significantly unchanged from about 1 hour after administration to at least about 24 hours after administration.

In a preferred aspect, the present invention is directed to a method of treating or preventing diseases associated with increased thrombotic risk due to platelet activation, aggregation or production comprising orally administering to a human in need thereof a controlled-release aspirin composition comprising ASA at an amount of about 325 milligrams, wherein the method provides a level of platelet aggregation inhibition within about 1 hour of administration and wherein the level of platelet aggregation inhibition remains significantly unchanged at least about 24 hours after administration and wherein the level of platelet aggregation inhibition is significantly increased 12 hours after administration as compared to 1 hour after administration.

In another preferred aspect, the present invention a method of treating or preventing diseases associated with increased thrombotic risk due to platelet activation, aggregation or production comprising orally administering to a human in need thereof a controlled-release aspirin composition comprising ASA at an amount from about 81 milligrams to about 325 milligrams, preferably from about 162.5 milligrams to 325 milligrams, wherein the method provides a level of platelet aggregation inhibition within about 1 hour of administration and wherein the level of platelet aggregation inhibition remains significantly unchanged from about 1 hour after administration to at least about 24 hours after administration and wherein the level of platelet aggregation inhibition is measured by a turbidimetric based optical detection system.

In another preferred aspect, the present invention a method of treating or preventing diseases associated with increased thrombotic risk due to platelet activation, aggregation or production comprising orally administering to a human in need thereof a controlled-release aspirin composition comprising ASA at an amount of about 325 milligrams, wherein the method provides a level of platelet aggregation inhibition within about 1 hour of administration and wherein the level of platelet aggregation inhibition remains significantly unchanged from about 1 hour after administration to at least about 24 hours after administration and wherein the level of platelet aggregation inhibition is significantly increased 16 hours after administration as compared to 1 hour after administration, and wherein the level of platelet aggregation inhibition is measured by a turbidimetric based optical detection system.

In another aspect, the present invention is directed to a method of reducing systemic or cardiovascular inflammation comprising orally administering to a human in need thereof a controlled-release aspirin comprising ASA at an amount from about 81 milligrams to about 325 milligrams, preferably from about 162.5 milligrams to 325 milligrams, wherein the method provides at least one of the following examples of systemic inflammation:
a) stimulation of vascular production of nitric oxide as measured by a significant change in mean reactive hyperemia index score as measured by pulse amplitude tonometry compared to a mean reactive hyperemia index score measured by pulse amplitude tonometry prior to oral administration of the composition to the human;
b) a significantly reduced high sensitive C-reactive protein level compared to a high sensitive C-reactive protein level after oral administration to a human of a bioequivalent amount of immediate-release aspirin, preferably 81 milligrams immediate-release aspirin; and
c) no significant change in interleukin-8 levels.

In another aspect, the present invention is directed to a method of preventing or treating cardiovascular disease comprising orally administering to a human in need thereof a controlled-release aspirin composition comprising ASA at an amount from about 81 milligrams to about 325 milligrams, preferably from about 162.5 milligrams to 325 milligrams, wherein the method provides greater than 100 nanograms of ASA per milliliter of serum for at least 4 hours, greater than 30 nanograms of ASA per milliliter of serum for at least 8 hours, more preferably greater than 1000 nanograms of salicylic acid per milliliter of serum for at least 8 hours, and most preferably greater than 200 nanograms of salicylic acid per milliliter of serum for at least 24 hours.

In another aspect, the present invention is directed to a method of preventing or treating cancer, preferably breast cancer or colorectal cancer, comprising orally administering to a human in need thereof a controlled-release aspirin composition comprising ASA at an amount from about 81 milligrams to about 325 milligrams, preferably from about 162.5 milligrams to 325 milligrams, wherein the method provides greater than 100 nanograms of ASA per milliliter of serum for at least 4 hours, greater than 30 nanograms of ASA per milliliter of serum for at least 8 hours, more preferably greater than 1000 nanograms of salicylic acid per milliliter of serum for at least 8 hours, and most preferably greater than 200 nanograms of salicylic acid per milliliter of serum for at least 24 hours.

In another aspect, the present invention is directed to a method of the invention wherein the controlled-release aspirin composition comprises crystalline ASA.

In another aspect, the present invention is directed to a method of the invention wherein the controlled-release aspirin composition comprises ASA and a coating agent, preferably the ratio of ASA to coating agent is at least 9 to 1, more preferably the coating agent comprises at least 84% ethylcellulose, about 4.7% povidone, about 7.0% castor oil and about 5.9% magnesium stearate wherein % is by weight. In a preferred aspect, the present invention is directed to a method of the invention comprise administering the controlled-release aspirin composition to a human with diabetes, preferably a human with diabetes and cardiovascular disease or at least two risk factors for cardiovascular disease. In a more preferred aspect the at least two risk factors for cardiovascular disease are selected from the group consisting of obesity, smoking, hypertension, hypercholesterolemia and history of thrombotic events whether or not on aspirin therapy.

In another aspect, the present invention is directed to a composition comprising an immediate release acetylsalicylic acid (IR) and an extended-release acetylsalicylic acid (ER) in a ratio from about 1:1 to about 6:1 IR:ER, preferably the IR:ER ratio is selected from the group 1:1, 1.5:1, 2:1, 2.5:1, 3:1, 3.5:1, 4:1, 4.5:1, 5:1, 5.5:1 or 6:1.

In another aspect, the present invention is directed to a method of platelet aggregation inhibition comprising orally administering to a human in need thereof a composition comprising an immediate release acetylsalicylic acid (IR) and an extended-release acetylsalicylic acid (ER) in a ratio from about 1:1 to about 6:1 IR:ER, preferably 1:1, 1.5:1, 2:1, 2.5:1, 3:1, 3.5:1, 4:1, 4.5:1, 5:1, 5.5:1 or 6:1, wherein platelet aggregation inhibition occurs within about 30 minutes to about 1 hour after administration and for at least 24 hours after administration.

In another aspect, the present invention is directed to a method of reducing serum thromboxane B2 levels comprising orally administering to a human in need thereof a composition comprising an immediate release acetylsalicylic acid (IR) and an extended-release acetylsalicylic acid (ER) in a ratio from about 1:1 to about 6:1 IR:ER, preferably 1:1, 1.5:1, 2:1, 2.5:1, 3:1, 3.5:1, 4:1, 4.5:1, 5:1, 5.5:1 or 6:1, wherein serum thromboxane B2 level reduction occurs within about 30 minutes to about 1 hour after administration and for at least 24 hours after administration.

In another aspect, the present invention is directed to a method of treating or preventing diseases associated with increased thrombotic risk due to platelet activation, aggregation or production comprising orally administering to a human in need thereof a composition comprising an immediate release acetylsalicylic acid (IR) and an extended-release acetylsalicylic acid (ER) in a ratio from about 1:1 to about 6:1 IR:ER, preferably 1:1, 1.5:1, 2:1, 2.5:1, 3:1, 3.5:1, 4:1, 4.5:1, 5:1, 5.5:1 or 6:1, wherein platelet aggregation inhibition occurs within about 30 minutes to about 1 hour after administration and for at least 24 hours after administration.

In another aspect, the present invention is directed to a method of reducing systemic or cardiovascular inflammation comprising orally administering to a human in need thereof a composition comprising an immediate release acetylsalicylic acid (IR) and an extended-release acetylsalicylic acid (ER) in a ratio from about 1:1 to about 6:1 IR:ER, preferably 1:1, 1.5:1, 2:1, 2.5:1, 3:1, 3.5:1, 4:1, 4.5:1, 5:1, 5.5:1 or 6:1, wherein systemic or cardiovascular inflammation reduction occurs within about 30 minutes to about 1 hour after administration and for at least 24 hours after administration.

In another aspect, the present invention is directed to a method of treating or preventing cardiovascular disease comprising orally administering to a human in need thereof a composition comprising an immediate release acetylsalicylic acid (IR) and an extended-release acetylsalicylic acid (ER) in a ratio from about 1:1 to about 6:1 IR:ER, preferably 1:1, 1.5:1, 2:1, 2.5:1, 3:1, 3.5:1, 4:1, 4.5:1, 5:1, 5.5:1 or 6:1, wherein for treatment of cardiovascular disease treatment occurs within about 30 minutes to about 1 hour after administration and for at least 24 hours after administration.

### BRIEF DESCRIPTION OF THE FIGURES

FIG. 1. Mean concentration-time profiles for ASA and SA after single dose extended release ASA (“ER ASA”; panels A and C) and immediate release ASA (“IR-ASA”; panels B and D).

FIG. 2. 24-hour inhibition of serum thromboxane (“TxB2”) production after single dose ER ASA and IR ASA.

FIG. 3. ID₅₀ of serum TxB2 production with single dose ER ASA and IR ASA.

FIG. 4. Platelet aggregation at 1, 12, 16 and 24 hours post 162.5 mg ER ASA (Durlaza®; Durlaza is a registered trademark of New Haven Pharmaceuticals, Inc.) and 325 mg ER ASA dosing. Upper lines are for collagen-induced platelet aggregation and lower lines are for arachidonic acid (“AA”) induced platelet aggregation.

FIG. 5. Effect of 162.5 mg ER ASA (Durlaza®) and 325 mg ER ASA on serum TxB2 levels.

FIG. 6. Effect of 162.5 mg ER ASA (Durlaza®) and 325 mg ER ASA on urinary 11-dehydroxythromboxane B2 and prostacyclin metabolite PGI₂-M levels.

FIG. 7. Effect of 162.5 mg ER ASA (Durlaza®) and 325 mg ER ASA on endothelial function as measured by Reactive Hyperemia Index (“RHI”).

FIG. 8. Mean high sensitive C-reactive protein (“hs-CRP”) by ASA dose. Error bars indicate standard error of the mean.

FIG. 9. Basal TxB2 and prostacyclin levels after aspirin dosing. * indicates statistically (p<0.05) different from IR ASA group. † indicates statistically (p<0.05) different from placebo. ‡ indicates statistically (p<0.05) different from 81 milligram dose.

FIG. 10. Normalized basal TxB2 and prostacyclin levels after aspirin dosing. * indicates statistically (p<0.05) different from IR ASA group.

FIG. 11. Bradykinin-stimulated TxB2 and prostacyclin levels after aspirin dosing. * indicates statistically (p<0.05) different from time 0. † indicates statistically (p<0.05) different from placebo. ‡ indicates statistically (p<0.05) different from 81 milligram dose.

FIG. 12. Bradykinin-stimulated IL-8 levels after aspirin dosing.

### DETAILED DESCRIPTION OF THE INVENTION

In one aspect, the present invention is directed to a controlled-release aspirin composition comprising acetylsalicylic acid (“ASA”) at an amount from about 162.5 milligrams to about 325 milligrams, wherein after oral administration to a human in need thereof the composition provides a level of platelet aggregation inhibition within about 1 hour of administration and wherein the level of platelet aggregation inhibition remains significantly unchanged from about 1 hour after administration to at least about 24 hours after administration.

Beyond ASA, salicylic acid has also been shown to be an anti-cancer agent. Specifically, salicylic acid has been shown to: decrease c-Myc levels expression in colon cancer (Ai G et al., Aspirin and salicylic acid decrease c-Myc expression in cancer cells: a potential role in chemoprevention, Tumour Biol. 2015 Aug. 28, Epub ahead of print); decrease cyclin A2 levels in cancer cell lines (Dachineni R et al., Cyclin A2 and CDK2 as Novel Targets of Aspirin and Salicylic Acid: A Potential Role in Cancer Prevention, Mol Cancer Res, 2016 March, 14(3), 241-252); and induce apoptosis in colon carcinoma cells (Zitta K et al., Salicylic acid induces apoptosis in colon carcinoma cells grown in-vitro: influence of oxygen and salicylic acid concentration, Exp Cell Res, 2012 Apr. 15, 318(7), 828-834.)

Not to be held to a particular theory, tumor growth also relies on pro-inflammatory mediators in the tumor micro-environment. The 24-hour release of anti-platelet aggregation and anti-inflammatory compounds ASA and salicylic acid, which is enabled by the present invention, is capable of providing around the clock inhibition of necessary components of cancer and cancer progression. This same around the clock inhibition is not afforded with IR-ASA. Thus, the ER-ASA compositions of the present invention and their methods of use are superior treatments for cancer.

### DEFINITIONS

As used herein, the term “composition” is intended to encompass a product comprising the specified ingredients in the specified amounts, as well as any product which results, directly or indirectly, from a combination of the specified ingredients in the specified amounts.

As used herein, all numerical values relating to amounts, weights, and the like, that are defined as “about” each particular value is plus or minus 10%. For example, the phrase “about 90% of the total composition” is to be understood as “81% to 99% of the total composition.” Therefore, amounts within 10% of the claimed value are encompassed by the scope of the claims.

As used herein, “from about” indicates the starting point of a range of numerical values with the lowest value in the range being 10% below the number being modified. For example, “from about 1% to about 10% of the total composition” describes a range with the lowest value being 0.9% and the highest value being 11%.

As used herein, the term “acetylsalicylic acid” is synonymous with “aspirin” and refers to

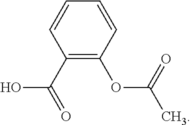 [SMILES: CC(=O)Oc1ccccc1C(=O)O]

a compound of the following formula:

As used herein, the term “salicylic acid” refers to a compound of the following formula:

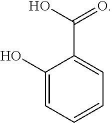

As used herein, the term “controlled-release” refers to the release of the active form of a drug after the administration and dissolution of the drug. Other forms of “controlled-release” are “extended release”, “delayed release”, “sustained release”, “prolonged release”, “programmed release”, “time release” and/or “rate controlled release”.

As used herein, the term “immediate-release” refers to the release of the active form of the drug upon administration or dissolution of the drug.

As used herein, the term “release” refers to making the active form of a drug available for chemical/pharmacodynamic interaction. For example, acetylsalicylic acid is available in its active form when it is capable of transferring its acetyl group to another chemical entity such as the mechanism for platelet inhibition.

As used herein, the term “oral administration” refers to administration of a composition of the invention to the mouth of a human for ingestion into the gastrointestinal tract.

As used herein, the term “Cₘₐₓ” refers to the maximum plasma concentration is determined by measuring the amount of the chemical entity per unit of plasma.

As used herein, the term “Tₘₐₓ” refers to the time to maximum plasma concentration.

As used herein, the term “AUC₀₋ₜ” refers to the area under the curve of a plot of the concentration of the chemical entity from time of administration to a defined timepoint (“t”) and is calculated by the trapezoidal rule.

As used herein, the term “AUCₗₐₛₜ” refers to the area under the curve of a plot of the concentration of the chemical entity from time of administration to the last measurable concentration and is calculated by the trapezoidal rule.

As used herein, the term “significant” or “significantly” refers to an amount that is statistically significant as calculated by statistical analysis of the data disclosed herein (i.e. paired t-test, sign rank test, etc. . . . )

As used herein, the term “prevent” or “preventing” refers to precluding, averting, obviating, forestalling, stopping, or hindering something from happening, especially by advance action.

As used herein, the term “treat” or “treating” refers to reversing, alleviating or slowing the progress of the disease, disorder, or condition to which such terms apply, or one or more symptoms of such disease, disorder, or condition.

As used herein, the term “inhibit” or “inhibiting” refers to the detectable reduction and/or elimination of a biological activity.

As used herein, the term “platelet aggregation” refers to clumping together of platelets in the blood or in vitro platelet-rich plasma samples.

As used herein, the term “disease associated with increased thrombotic risk due to platelet activation, aggregation or production” refers to a disease in which the subject suffering from that disease has an increased risk of a thrombotic event over a subject not suffering from that disease. Diseases associated with increased thrombotic risk include, but are not limited to, essential thrombocytosis, essential thrombocythemia, associated myeloid, myelofibrotic or myeloproliferative diseases.

As used herein, the term “thrombotic event” refers to an event caused by the formation of a blood clot inside a blood vessel of a subject that obstructs the flow of blood through the circulatory system of the subject.

Coating agents suitable for use in the present invention include, but are not limited to, polymers, lubricants, plasticizers, stabilizing agents and combinations thereof. In a preferred embodiment the coating agent is a combination of polymers, lubricants, plasticizers and stabilizing agents. In a more preferred embodiment the coating agent is from about 1% to about 10% of the total composition or about 8% to about 10% of the total composition. In an even more preferred embodiment the coating agent is about 8% or about 9% of the total composition.

Anti-caking agents suitable for use in the present invention include, but are not limited to, colloidal anhydrous silica, talc and combinations thereof. In a preferred embodiment the anti-caking agent is a combination of colloidal anhydrous silica and talc. In a more preferred embodiment the anti-caking agent is from about 1% to about 5% of the total composition. In a more preferred embodiment the anti-caking agent is about 3.5% of the total composition.

Film forming polymers that are insoluble in the gastrointestinal environment (“P1”) and are suitable for use in the present invention include, but are not limited to, zein, ethylcellulose, vinyl chloride, vinyl acetate and/or its copolymers, copolymers based on ethyl and/or methyl acrylate and/or methacrylate, for example the products marketed under the trademark EUDRAGIT® RL and/or RS (EUDRAGIT is a registered trademark of Evonik Roehm GMBH), and mixtures of thereof. In preferred embodiments P1 is ethylcellulose. In more preferred embodiments ethylcellulose is at a concentration of about 74% of the coating agent.

Water soluble polymers (“P2”) suitable for use in the present invention include, but are not limited to, povidine (i.e. polyvinylpyrrolidone), water-soluble cellulose derivatives such as hydroxypropyl cellulose, hydroxypropyl methyl cellulose, hydroxymethyl ethyl cellulose and methyl cellulose, vinyl acetate/crotonic acid copolymers, maleic anhydride/methyl vinyl ether copolymers, and derivatives and combinations thereof. In a preferred embodiment P2 is povidone. In a more preferred embodiment povidone is at a concentration of about 4% of the coating agent.

Lubricants suitable for use in the present invention include, but are not limited to, alkaline earth metal salts of stearic acid, magnesium silicates, kaolin, talc, silica and combinations thereof. In a preferred embodiment the lubricant is magnesium stearate. In a more preferred embodiment magnesium stearate is at a concentration of about 5% of the coating agent.

Plasticizers suitable for use in the present invention include, but are not limited to, stearates of a glycol such as glycerol, propylene glycol or triacetin, citrates, phthalates, for example dimethyl, diethyl or dibutyl phthalate, esters of cetyl alcohol, such as cetyl palmitate in particular, sebacates, tartrates, castor oil, cutin, synthetic resins, for example Cérit, and combinations thereof. In a preferred embodiment the plasticizer is castor oil. In a more preferred embodiment castor oil is at a concentration of about 6% of the coating agent.

Stabilizing agents suitable for use in the present invention include, but are not limited to tartaric acid. In a more preferred embodiment tartaric acid is at a concentration of about 10% of the coating agent.

Cardiovascular diseases include, but are not limited to, cardiac arrhythmia, vascular disease, myocardial infarction, unstable angina, congestive heart failure, myocarditis, atherosclerosis, and restenosis.

The term “cancer,” as used herein, refers to any disease that results from the uncontrolled division of cells capable of metastasizing.

Cancer includes, but is not limited to, cancer types including breast cancer including male breast cancer; digestive/gastrointestinal cancers including anal cancer, appendix cancer, extrahepatic bile duct cancer, gastrointestinal carcinoid tumor, colon cancer, esophageal cancer, gallbladder cancer, gastric cancer, gastrointestinal stromal tumors (“gist”), Islet cell tumors, adult primary liver cancer, childhood liver cancer, pancreatic cancer, rectal cancer, small intestine cancer, and stomach (gastric) cancer; endocrine and neuroendocrine cancers including pancreatic adenocarcinoma, adrenocortical carcinoma, pancreatic neuroendocrine tumors, Merkel cell carcinoma, non-small cell lung neuroendocrine tumor, small cell lung neuroendocrine tumor, parathyroid cancer, pheochromocytoma, pituitary tumor and thyroid cancer; eye cancers including intraocular melanoma and retinoblastoma; genitourinary cancer including bladder cancer, kidney (renal cell) cancer, penile cancer, prostate cancer, transitional cell renal pelvis and ureter cancer, testicular cancer, urethral cancer and Wilms tumor; germ cell cancers including childhood central nervous system cancer, childhood extracranial germ cell tumor, extragonadal germ cell tumor, ovarian germ cell tumor and testicular cancer; gynecologic cancers including cervical cancer, endometrial cancer, gestational trophoblastic tumor, ovarian epithelial cancer, ovarian germ cell tumor, uterine sarcoma, vaginal cancer and vulvar cancer; head and neck cancers including hypopharyngeal cancer, laryngeal cancer, lip and oral cavity cancer, metastatic squamous neck cancer with occult primary, mouth cancer, nasopharyngeal cancer, oropharyngeal cancer, paranasal sinus and nasal cavity cancer, parathyroid cancer, pharyngeal cancer, salivary gland cancer and throat cancer; leukemias including adult acute lymphoblastic leukemia, childhood acute lymphoblastic leukemia, adult acute myeloid leukemia, childhood acute myeloid leukemia, chronic lymphocytic leukemia, chronic myelogenous leukemia and hairy cell leukemia; lymphomas including AIDS-related lymphoma, cutaneous t-cell lymphoma, adult Hodgkin lymphoma, childhood Hodgkin lymphoma, Hodgkin lymphoma during pregnancy, mycosis fungoides, adult non-Hodgkin lymphoma, childhood non-Hodgkin lymphoma, non-Hodgkin lymphoma during pregnancy, primary central nervous system lymphoma, Sézary syndrome and Waldenström macroglobulinemia; musculoskeletal cancers including Ewing sarcoma, osteosarcoma and malignant fibrous histocytoma of bone, childhood rhabdomyosarcoma and soft-tissue sarcoma; neurological cancers including adult brain tumor, childhood brain tumor, astrocytomas, brain stem glioma, central nervous system atypical teratoid/rhabdoid tumor, central nervous system embryonal tumors, craniopharyngioma, ependymoma, neuroblastoma, primary central nervous system (CNS) lymphoma; respiratory/thoracic cancers including non-small cell lung cancer, small cell lung cancer, malignant mesothelioma, thymoma and thymic carcinoma; and skin cancers including Kaposi sarcoma, melanoma and squamous cell carcinoma.

The following representative embodiments are provided solely for illustrative purposes and are not meant to limit the invention in any way.

### Preferred Composition Embodiments

Preferred composition embodiments of the present invention include:

a controlled-release aspirin composition comprising:

about 162.5 mg acetylsalicylic acid;

about 12.322 mg ethylcellulose;

about 0.695 mg povidone;

about 1.04 mg castor oil;

about 0.886 mg magnesium stearate; and

about 1.694 mg tartaric acid;

a controlled-release aspirin composition comprising:

about 162.5 mg acetylsalicylic acid;

about 12.322 mg ethylcellulose;

about 0.695 mg povidone;

about 1.04 mg castor oil;

about 0.886 mg magnesium stearate;

about 1.694 mg tartaric acid;

about 4.641 mg colloidal anhydrous silica; and

about 1.856 mg talc;

a controlled-release aspirin composition comprising:

about 325 mg acetylsalicylic acid;

about 24.644 mg ethylcellulose;

about 1.39 mg povidone;

about 2.08 mg castor oil;

about 1.772 mg magnesium stearate; and

about 3.388 mg tartaric acid; and

a controlled-release aspirin composition comprising:

about 325 mg acetylsalicylic acid;

about 24.644 mg ethylcellulose;

about 1.39 mg povidone;

about 2.08 mg castor oil;

about 1.772 mg magnesium stearate;

about 3.388 mg tartaric acid;

about 9.282 mg colloidal anhydrous silica; and

about 3.712 mg talc.

Further preferred composition embodiments include:

a controlled-release aspirin composition comprising ASA at a concentration of from about 90% to about 99% of the total composition and a coating agent at a concentration of from about 1% to about 10% of the total composition, wherein the composition is in the form of a microcapsule and wherein after oral administration to a human in need thereof, the composition provides:
a mean time to maximum plasma concentration (Tₘₐₓ) of ASA of about 2.4±1.2 hours when the composition provides a 325 mg dose and about 1.9±1.2 hours when the composition provides a 162.5 mg dose;
a mean maximum plasma concentration (Cₘₐₓ) of ASA of about 401±124 nanograms/milliliters when the composition provides a 325 mg dose and about 174±46 nanograms/milliliters when the composition provides a 162.5 mg dose;
a mean area under the plasma concentration time curve from time zero to the last concentration (AUCₗₐₛₜ) of ASA of about 1550±521 nanograms/milliliters*hours when the composition provides a 325 mg dose and about 599±276 nanograms/milliliters*hours when the composition provides a 162.5 mg dose; and/or
a mean area under the plasma concentration time curve from time zero to eight hours (AUC₀₋₈) of ASA of about 1680±517 nanograms/milliliters*hours when the composition provides a 325 mg dose and about 845±122 nanograms/milliliters*hours when the composition provides a 162.5 mg dose;
   a controlled-release aspirin composition comprising ASA at a concentration of from about 90% to about 99% of the total composition and a coating agent at a concentration of from about 1% to about 10% of the total composition, wherein the composition is in the form of a microcapsule and wherein after oral administration to a human in need thereof, the composition provides a Cₘₐₓ of ASA of about 401±124 nanograms/milliliters when the composition provides a 325 mg dose and about 174±46 nanograms/milliliters when the composition provides a 162.5 mg dose and a plasma concentration that is greater than about 14% of the Cₘₐₓ for about 8 hours when the composition provides a 325 mg dose and greater than about 17% of the mean Cₘₐₓ for about 8 hours when the composition provides a 162.5 mg dose;
   a controlled-release aspirin composition comprising ASA at a concentration of from about 90% to about 99% of the total composition and a coating agent at a concentration of from about 1% to about 10% of the total composition, wherein the composition is in the form of a microcapsule and wherein after oral administration to a human in need thereof, the composition provides:
a Tₘₐₓ of salicylic acid of about 4.8±1.5 hours when the composition provides a 325 mg dose and about 4.5±1.2 hours when the composition provides a 162.5 mg dose;
a Cₘₐₓ of salicylic acid of about 5330±1732 nanograms/milliliters when the composition provides a 325 mg dose and about 2280±882 nanograms/milliliters when the composition provides a 162.5 mg dose;
an AUCₗₐₛₜ of salicylic acid of about 55700±22224 nanograms/milliliters*hours when the composition provides a 325 mg dose and about 22300±8095 nanograms/milliliters*hours when the composition provides a 162.5 mg dose; and/or
an AUC₀₋₈ of salicylic acid of about 57200±22308 nanograms/milliliters*hours when the composition provides a 325 mg dose and about 23900±7266 nanograms/milliliters*hours when the composition provides a 162.5 mg dose; and
   a controlled-release aspirin composition comprising ASA at a concentration of from about 90% to about 99% of the total composition and a coating agent at a concentration of from about 1% to about 10% of the total composition, wherein the composition is in the form of a microcapsule and wherein after oral administration to a human in need thereof, the composition provides a Cₘₐₓ of salicylic acid of about 5330±1732 nanograms/milliliters when the composition provides a 325 mg dose and about 2280±882 nanograms/milliliters when the composition provides a 162.5 mg dose and a plasma concentration that is greater than about 16% of the mean Cₘₐₓ for about 24 hours when the composition provides a 325 mg dose and greater than about 13% of the mean Cₘₐₓ for about 24 hours when the composition provides a 162.5 mg dose.

The following preferred embodiments are provided solely for illustrative purposes and are not meant to limit the invention in any way.

### Preferred Method Embodiments

Preferred method embodiments of the invention include:

a method of inhibiting platelet aggregation comprising orally administering to a human in need thereof a controlled-release aspirin composition comprising acetylsalicylic acid (“ASA”) at an amount from about 81 milligrams to about 325 milligrams, preferably from about 162.5 milligrams to 325 milligrams, wherein the method provides a level of platelet aggregation inhibition within about 1 hour of administration and wherein the level of platelet aggregation inhibition remains significantly unchanged from about 1 hour after administration to at least about 24 hours after administration;

a method of reducing serum thromboxane B2 levels comprising orally administering to a human in need thereof a controlled-release aspirin composition comprising ASA at an amount from about 81 milligrams to about 325 milligrams, preferably from about 162.5 milligrams to 325 milligrams, wherein the method provides a reduced serum thromboxane B2 level within 1 hour of administration and wherein the serum thromboxane level remains significantly unchanged from about 1 hour after administration to at least about 24 hours after administration, preferably the method provides no significant change in urinary levels of thromboxane B2;

a method of reducing serum thromboxane B2 levels thereof comprising orally administering to a human in need thereof a controlled-release aspirin composition comprising ASA at an amount of about 81 milligrams, wherein the method provides a reduced serum thromboxane B2 level within 1 hour of administration and wherein the serum thromboxane level remains significantly unchanged from about 1 hour after administration to at least about 24 hours after administration and wherein the method provides no significant change in urinary levels of 2,3-dinor-6-keto-PGF1α;

a method of reducing serum thromboxane B2 levels comprising orally administering to a human in need thereof a controlled-release aspirin composition comprising ASA at an amount of about 162.5 milligrams, wherein the method provides a reduced serum thromboxane B2 level within 1 hour of administration and wherein the serum thromboxane level remains significantly unchanged from about 1 hour after administration to at least about 24 hours after administration and wherein the method provides lower serum thromboxane B2 levels than after administration of the controlled-release aspirin composition comprising about 81 milligrams ASA;

a method of treating or preventing diseases associated with increased thrombotic risk due to platelet activation, aggregation or production comprising orally administering to a human in need thereof a controlled-release aspirin composition comprising acetylsalicylic acid (“ASA”) at an amount from about 81 milligrams to about 325 milligrams, preferably from about 162.5 milligrams to 325 milligrams, wherein the method provides a level of platelet aggregation inhibition within about 1 hour of administration and wherein the level of platelet aggregation inhibition remains significantly unchanged from about 1 hour after administration to at least about 24 hours after administration;

a method of reducing systemic or cardiovascular inflammation comprising orally administering to a human in need thereof a controlled-release aspirin comprising ASA at an amount from about 81 milligrams to about 325 milligrams, preferably from about 162.5 milligrams to 325 milligrams, wherein the method provides at least one of:
a) a significant change in mean reactive hyperemia index score as measured by pulse amplitude tonometry compared to a mean reactive hyperemia index score measured by pulse amplitude tonometry prior to oral administration of the composition to the human;
b) a significantly reduced high sensitive C-reactive protein level compared to a high sensitive C-reactive protein level after oral administration to a human of a bioequivalent amount of immediate-release aspirin, preferably 81 milligrams immediate-release aspirin; and
c) no significant change in interleukin-8 levels;

a method of preventing or treating cardiovascular disease comprising orally administering to a human in need thereof a controlled-release aspirin composition comprising ASA at an amount from about 81 milligrams to about 325 milligrams, preferably from about 162.5 milligrams to 325 milligrams, wherein the method provides greater than 100 nanograms of ASA per milliliter of serum for at least 4 hours, greater than 30 nanograms of ASA per milliliter of serum for at least 8 hours, more preferably greater than 1000 nanograms of salicylic acid per milliliter of serum for at least 8 hours, and most preferably greater than 200 nanograms of salicylic acid per milliliter of serum for at least 24 hours; and

a method of preventing or treating cancer, preferably breast cancer or colorectal cancer, comprising orally administering to a human in need thereof a controlled-release aspirin composition comprising ASA at an amount from about 81 milligrams to about 325 milligrams, preferably from about 162.5 milligrams to 325 milligrams, wherein the method provides greater than 100 nanograms of ASA per milliliter of serum for at least 4 hours, greater than 30 nanograms of ASA per milliliter of serum for at least 8 hours, more preferably greater than 1000 nanograms of salicylic acid per milliliter of serum for at least 8 hours, and most preferably greater than 200 nanograms of salicylic acid per milliliter of serum for at least 24 hours.

Immediate Release ASA and Extended Release ASA Composition

Not to be held to a particular theory, it is believed that the combination of an immediate-release ASA (“IR ASA”) and extended-release ASA (“ER ASA”) will provide an effect that eliminates the slow onset of an extended-release ASA while also eliminating the short duration of effect for an immediate-release ASA. This effect is due in part to the different gastrointestinal dissolution profiles of IR ASA and ER ASA. Specifically, oral administration of IR ASA leads to a rapid absorption in the gastrointestinal tract whereas oral administration of ER ASA leads to prolonged absorption resulting in sustained blood levels. The following representative embodiments are not meant to limit the invention in any way.

Preferred compositions include:

a composition comprising an immediate release acetylsalicylic acid (IR) and an extended-release acetylsalicylic acid (ER) in a ratio from about 1:1 to about 6:1 IR:ER, preferably the IR:ER ratio is selected from the group consisting of 1:1, 1.5:1, 2:1, 2.5:1, 3:1, 3.5:1, 4:1, 4.5:1, 5:1, 5.5:1 and 6:1.

Preferred methods utilizing an IR ASA and ER ASA composition include:

a method of platelet aggregation inhibition comprising orally administering to a human in need thereof a composition comprising an immediate release acetylsalicylic acid (IR) and an extended-release acetylsalicylic acid (ER) in a ratio from about 1:1 to about 6:1 IR:ER, preferably 1:1, 1.5:1, 2:1, 2.5:1, 3:1, 3.5:1, 4:1, 4.5:1, 5:1, 5.5:1 or 6:1;
a method of reducing serum thromboxane B2 levels comprising orally administering to a human in need thereof a composition comprising an immediate release acetylsalicylic acid (IR) and an extended-release acetylsalicylic acid (ER) in a ratio from about 1:1 to about 6:1 IR:ER, preferably 1:1, 1.5:1, 2:1, 2.5:1, 3:1, 3.5:1, 4:1, 4.5:1, 5:1, 5.5:1 or 6:1;
a method of reducing systemic or cardiovascular inflammation comprising orally administering to a human in need thereof a composition comprising an immediate release acetylsalicylic acid (IR) and an extended-release acetylsalicylic acid (ER) in a ratio from about 1:1 to about 6:1 IR:ER, preferably 1:1, 1.5:1, 2:1, 2.5:1, 3:1, 3.5:1, 4:1, 4.5:1, 5:1, 5.5:1 or 6:1; and
a method of treating or preventing cardiovascular disease comprising orally administering to a human in need thereof a composition comprising an immediate release acetylsalicylic acid (IR) and an extended-release acetylsalicylic acid (ER) in a ratio from about 1:1 to about 6:1 IR:ER, preferably 1:1, 1.5:1, 2:1, 2.5:1, 3:1, 3.5:1, 4:1, 4.5:1, 5:1, 5.5:1 or 6:1.

The following Examples are provided solely for illustrative purposes and are not meant to limit the invention in any way.

### EXAMPLES

### Example 1—Pharmacokinetics of IR ASA vs. ER ASA

Extended release acetylsalicylic acid (“ER ASA”) compositions including 162.5 mg ER ASA (Durlaza®) demonstrate a delayed Tₘₐₓ a lower Cₘₐₓ and a greater AUC than immediate release acetylsalicylic acid (“IR ASA”). Additionally, ER ASA compositions maintain Cₘₐₓ for a longer period of time and remain at therapeutic levels in blood serum longer than IR ASA. Surprisingly, a dose of 162.5 mg ER ASA (Durlaza®) is needed to provide similar platelet activity to 81 mg IR ASA.

### Methods

Healthy adults were randomized to receive single doses of extended release ASA (“ER ASA”) at a dosage of 20 mg, 40 mg, 81 mg, 162.5 mg ER ASA (Durlaza®; New Haven Pharmaceuticals, Inc., North Haven, Conn.), or 325 mg, or immediate release ASA (“IR ASA”; aspirin powder USP; Letco Medical, Decatur, Ala.) at a dosage of 5 mg, 10 mg, 20 mg, 40 mg or 81 mg. For pharmacokinetic assessments (Cₘₐₓ, Tₘₐₓ area under the curve [AUC], and AUCₗₐₛₜ), serial blood samples were taken within 1 hour before dosing and at 0.25, 0.5, 1, 1.5, 2, 4, 8, 16, and 24 hours after drug administration. For pharmacodynamic assessments, serum 11-dehydro-thromboxane (“TxB2”, a stable metabolite of thromboxane A2) levels were assessed using a validated enzyme immunoassay kit in serial blood samples collected 1 hour before dosing and at 0.5, 1, 2, 4, 8, 16, and 24 hours after drug administration.

### Results

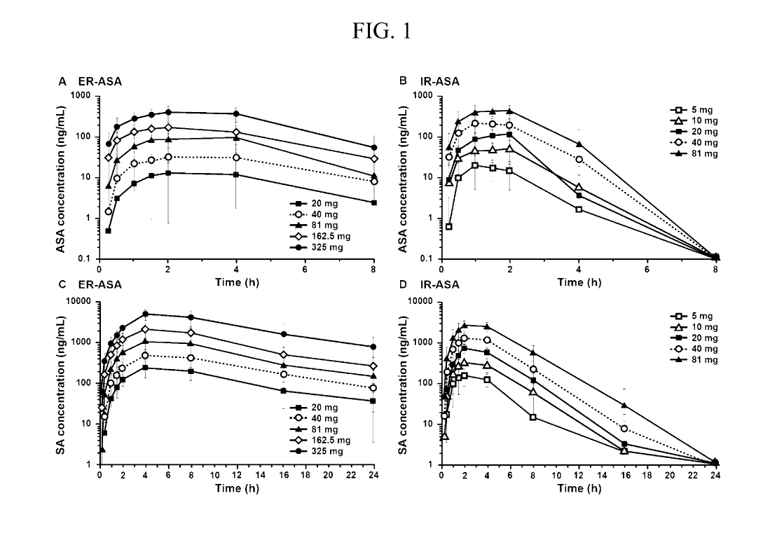

Changes in Cₘₐₓ and AUCₗₐₛₜ for ASA were dose-proportional. The time required to reach the maximum concentration of ASA was slightly delayed with all doses of ER ASA (2-3 hours) compared with IR ASA (1-2 hours). See Table 1. Maximum serum concentrations of ASA and the deacetylated salicylic acid (“SA”) were lower with ER ASA compared with IR ASA but were maintained over a longer time period (i.e. for ASA through the 8 hours evaluated in the study [sampling interval] with ER ASA vs. 6 hours with IR ASA). See FIG. 1, panel A vs. B.

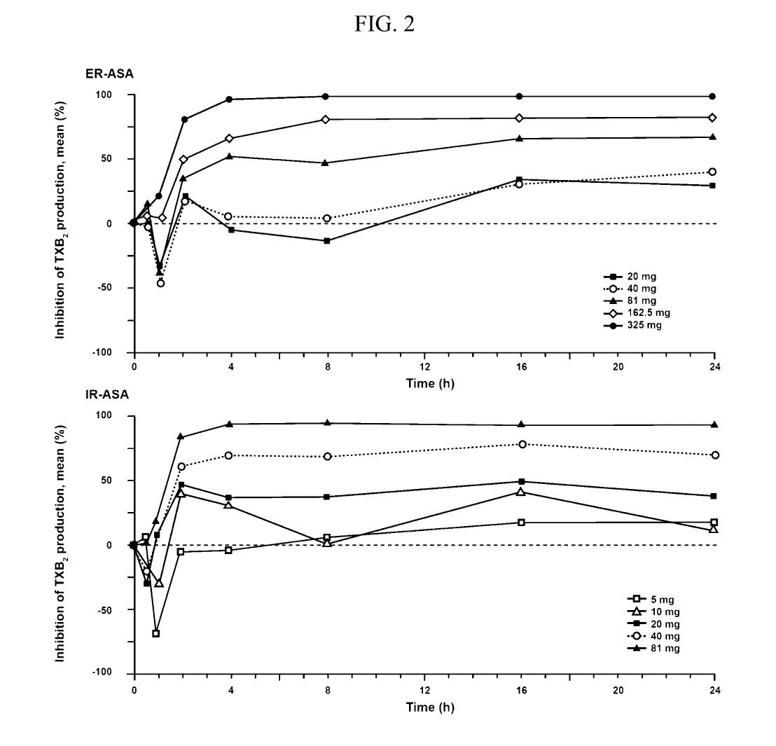

Further, single dose inhibition of TxB2 production was maintained during a 24-hour period with the highest doses of ER ASA (i.e. 81 mg, 162.5 mg (Durlaza®), and 325 mg) and IR ASA (i.e. 40 mg and 81 mg). See FIG. 2.

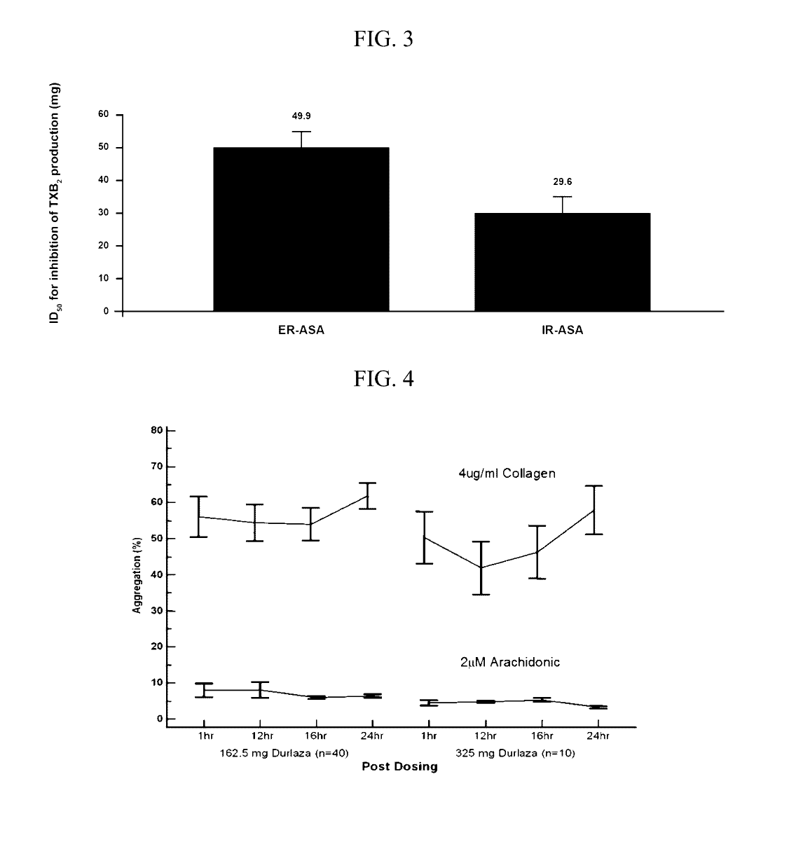

Finally, a ˜2-fold greater dose of ER ASA (162.5 mg, Durlaza®) was required to achieve a 50% maximum inhibition (“ID₅₀”) of serum TxB2 production that was similar to that of IR ASA 81 mg. See FIG. 3. This suggests, for a single dose, that ˜2-fold greater dose of ER ASA (162.5 mg/d), which is still considered a low dose, is needed to provide bioequivalent antiplatelet activity to that of IR ASA 81 mg/d. There was no previously known or established ratio of ER:IR ASA for single-dose TxB2 inhibition.

TABLE 1
Single Dose ASA Pharmacokinetics

| | | | | |
|---|---|---|---|---|
| | | Cₘₐₓ, | AUC_{0-8,} | AUC_{last,} |
| | T_{max,} h | ng/mL | ng/mL · hr | ng/mL · hr |
| Dose | (SD) | (SD) | (SD) | (SD) |

ER ASA

| 20 | mg | 1.9 | (1.6) | 18.6 | (7.2) | 133 | (NC) | 22.9 | (39.6) |
|---|---|---|---|---|---|---|---|---|---|
| 40 | mg | 2.8 | (2.0) | 38.6 | (11.0) | 213 | (62) | 107 | (83) |
| 81 | mg | 2.7 | (1.3) | 106 | (47) | 439 | (107) | 322 | (122) |
| 162.5 mg | | 1.9 | (1.2) | 174 | (46) | 845 | (122) | 599 | (276) |
| (Durlaza ®) | | | | | | | | | |
| 325 | mg | 2.4 | (1.2) | 401 | (124) | 1680 | (517) | 1550 | (521) |

IR ASA

| 5 | mg | 1.2 | (0.6) | 25.1 | (8.7) | NC | | 26.8 | (9.9) |
|---|---|---|---|---|---|---|---|---|---|
| 10 | mg | 1.5 | (0.9) | 63.9 | (29.1) | 136 | (NC) | 74.9 | (30.4) |
| 20 | mg | 1.6 | (0.4) | 123 | (29) | 366 | (NC) | 148 | (55) |

| | | | | | | | | |
|---|---|---|---|---|---|---|---|---|
| 40 | mg | 1.4 | (0.6) | 228 | (79) | NC | 384 | (118) |
| 81 | mg | 1.4 | (0.6) | 504 | (136) | NC | 865 | (248) |

ER ASA (SA)

| 20 | mg | 4.8 | (1.5) | 262 | (86) | 2930 | (609) | 2560 | (735) |
|---|---|---|---|---|---|---|---|---|---|
| 40 | mg | 4.8 | (1.5) | 531 | (127) | 5780 | (1798) | 5540 | (1856) |
| 81 | mg | 4.9 | (2.0) | 1240 | (486) | 12900 | (3947) | 12100 | (4175) |
| 162.5 mg | | 4.5 | (1.2 | 2280 | (882) | 23900 | (7266) | 22300 | (8095) |
| (Durlaza ®) | | | | | | | | | |
| 325 | mg | 4.8 | (1.5) | 5330 | (1732) | 57200 | (22308) | 55700 | (22224) |

IR ASA (SA)

| | | | | | | | | | |
|---|---|---|---|---|---|---|---|---|---|
| 5 | mg | 2.3 | (1.0) | 188 | (45) | 1650 | (NC) | 524 | (220) |
| 10 | mg | 2.5 | (1.0) | 417 | (90) | 1790 | (379) | 1450 | (496) |
| 20 | mg | 2.3 | (0.7) | 799 | (136) | 3390 | (1349) | 3140 | (788) |
| 40 | mg | 2.6 | (1.0) | 1500 | (288) | 7090 | (2212) | 6200 | (1606) |
| 81 | mg | 2.7 | (1.3) | 3150 | (671) | 16300 | (4743) | 1400 | (412) |

NC = not calculable

### Example 2—Effect of 162.5 mg ER ASA (Durlaza®) and 325 mg ER ASA on Platelet Inhibitory Effect in Patients with Type II Diabetes and a History of Cardiovascular Disease

First, platelet aggregation inhibition was maintained over the entire 24 hour dosing period. A single dose of 162.5 mg ER ASA (Durlaza®) is capable of maintaining a level of platelet aggregation inhibition starting at 1 hour post administration without a significant loss of the inhibition effect through 24 hours post administration. In patients with high platelet turnover or high platelet reactivity, a dose of 325 mg ER ASA is similarly capable of maintaining platelet aggregation inhibition for 24 hours without a significant loss of the inhibition effect. Further, administration of 325 mg ER ASA resulted in a significant increase in platelet aggregation inhibition at 24 hours post administration indicating a dose sensitive response. Finally, neither 162.5 mg ER ASA (Durlaza®) nor 325 mg ER ASA had a significant loss of effect as measured in aspirin reactive units (“ARU”) compared to 1 hour post-administration indicating a continued therapeutic benefit over 24 hours.

Second, ER ASA compositions reduced serum TxB2 levels but not urinary TxB2 or prostacyclin levels. Both 162.5 mg ER ASA (Durlaza®) and 325 mg ER ASA surprisingly lowered blood serum levels of TxB2 over a 24 hour post administration period. Further, administration of 325 mg ER ASA resulted in significantly lower blood serum levels of TxB2 as compared to 162.5 mg ER ASA (Durlaza®) indicating a dose sensitive response. Finally, neither 162.5 mg ER ASA (Durlaza®) nor 325 mg ER ASA had a significant effect on the level of either urinary 11-deydroxythromboxane B2 nor prostacyclin metabolite PGI₂-M. This indicates that the majority of ASA from the ER ASA compositions is deacetylated in the liver.

Third, ER ASA compositions exhibited anti-inflammatory properties. 325 mg ER ASA and not 162.5 mg ER ASA (Durlaza®) improved the patient's reactive hyperemia index (“RHI”) score as compared to 81 mg IR aspirin. This indicates that 325 mg ER ASA may exhibit anti-inflammatory properties. Further, 162.5 mg ER ASA (Durlaza®) therapy lowered high-sensitive C-reactive protein (“hs-CRP”) levels as compared to 81 mg IR aspirin and remained lower with 325 mg ER ASA. There was no difference between CRP level between pre- and 24 hour post-measurements with 162.5 mg ER ASA (Durlaza®) and 325 mg ER ASA.

### Overall Methods

40 subjects were enrolled in a study (main and sub-study) to assess the change in platelet inhibitory effect of Durlaza® in cardiovascular disease subjects at risk of demonstrating high platelet turnover during the course of a 24-hour dosing interval. Specifically, these subjects had a history of Type II diabetes and vascular disease including coronary artery disease (“CAD”), peripheral vascular disease (“PVD”), and cardiovascular disease (“CVD”) or multiple CVD risk factors such as obesity, current smoking, hypertension, hypercholesterolemia and history of thrombotic event whether or not on aspirin therapy. High platelet reactivity has previously been implicated in incomplete inhibition during therapy with immediate release low-dose aspirin in patients with type II diabetes.

The main study consisted of a run-in phase where subjects were administered 81 mg, everyday (“QD”), of immediate release acetylsalicylic acid (“IR ASA”) for 7-28 days. Next, subjects were administered 162.5 mg ER ASA, QD, (Durlaza®) for 14±4 days. Of the 40 subjects that participated in the main study, 10 subjects that exhibited high platelet turnover (“HPT”) were selected to participate in the sub-study. HPT was determined by meeting at least two of the following criteria: an immature platelet fraction of ≧3.0, a mean platelet volume of ≧11 fl, a VerifyNow® aspirin reaction units (“ARU”) of ≧550 (VerifyNow is a registered trademark of Accumetrics, Inc.), a 2 micromolar (“μM”) arachidonic acid (“AA”) (max value)-induced light transmittance aggregometry (“LTA”)≧10%, a 4 microgram per milliliter (“μg/mL”)-collagen induced LTA≧70% and Multiplate® arbitrary units (i.e. adenosine diphosphate) of ≧30 (Multiplate is a registered trademark of Roche Diagnostics GMBH).

In the sub-study, subjects were administered 81 mg, QD, of IR ASA for approximately 14 days. Next, subjects were administered 325 mg ER ASA, QD, for 14±4 days.

A pre-screen visit (Visit 1) was conducted for all subjects of the study. The main study consisted of 3 visits (Visits 2-4) including a dosing visit (Visit 2), an in-patient housing visit (Visit 3) and a telephone call made to participants for safety follow-up (Visit 4). The 325 mg ER ASA sub-study consisted of 3 visits (Visits 5-7) which corresponded with Visits 2-4 of the main study.

During Visits 2 and 5 subjects were subjected to the following tests: (1) platelet aggregation measurements (a) AA induced, (b) collagen induced, and (c) Multiplate®; (2) urinary TxB2, (3) urinary prostacyclin metabolite (“PGI-M”), (4) serum TxB2, (5) platelet turnover measurements, (6) endothelial function measurement, and (7) aspirin esterase activity measurements.

During Visits 3 and 6 subjects were subjected to the following tests: (1) platelet aggregation measurements (a) AA induced, (b) collagen induced, and (c) Multiplate®; (2) urinary TxB2), (3) urinary PGI-M, (4) serum TxB2, and (5) endothelial function measurement.

Subjects were also measured for high sensitive C-reactive protein (“hs-CRP”) levels at Visit 1, Visit 2, Visit 3 pre-dose, Visit 3 at 24 hours, Visit 6 pre-dose and Visit 6 at 24 hours.

### I. Platelet Aggregation Measurements

A. AA and Collagen-Induced Platelet Aggregation Inhibition

The change in platelet aggregation (“PA”) over 24 hours (“PA₂₄”) using data from 1 and 2 micromolar (“μM”) AA, and 2 and 4 microgram/ml (“μg/mL”) collagen were calculated using the formulas: change PA₁₂=(PA₁₂−PA₁); change PA₁₆=(PA₁₆−PA₁ and change PA₂₄=(PA₂₄−PA₁).

1. Visit-3 162.5 mg ER ASA (Durlaza®)

An increase in platelet aggregation (ΔPA₁₂, or ΔPA₁₆ or ΔPA₂₄) by more than 15% with either agonists (i.e., arachidonic acid or collagen) is considered a significant loss of aspirin effect over 24 hours. As shown in Table 2 there was no significant change of the antiplatelet effect at 12, 16 or 24 hours versus hour 1 after administration of 162.5 mg ER ASA (Durlaza®) following 14±4 days of QD administration. It was impressive and unknown whether a single dose of ER ASA could establish and maintain complete (less than 15% increase per the protocol) platelet inhibition over the entire course of a 24 hour dosing interval, with no increase in platelet aggregation at 24 hours compared to hour 1.

TABLE 2
Change in Platelet Aggregation after Administration
of 162.5 mg ER ASA (Durlaza ®)

| | | | | |
|---|---|---|---|---|
| 162.5 mg ER ASA | | | | |
| (Durlaza ®) post dose | 1 h | 12 h | 16 h | 24 h |

LTA (Max %)

| | | | | |
|---|---|---|---|---|
| 1 mM Arachidonic Acid | 5 ± 12 | 5 ± 11 | 4 ± 3 | 3 ± 2 |
| 2 mM Arachidonic Acid | 7 ± 13 | 8 ± 12 | 6 ± 3 | 6 ± 3 |
| 2 ug/ml Collagen | 48 ± 25 | 50 ± 25 | 43 ± 21 | 40 ± 22 |
| 4 ug/ml Collagen | 51 ± 21 | 55 ± 28 | 55 ± 23 | 62 ± 20 |

Multiplate ® (AU*Min)

| | | | | |
|---|---|---|---|---|
| Arachidonic Acid | 17 ± 12 | 16 ± 13 | 16 ± 13 | 18 ± 11 |
| Collagen | 33 ± 17 | 34 ± 19 | 32 ± 20 | 35 ± 17 |

2. Visit-6 325 mg ER ASA

As shown in Table 3 there was also not a significant change of the antiplatelet effect at 12, 16 or 24 hours versus hour 1 after administration of 325 mg ER ASA following 14±4 days of QD administration as measured by AA-induced platelet aggregation (1 mM or 2 mM) or 2 μg/mL collagen induced platelet aggregation.

However, as shown in Table 3 there was a significant increase of the antiplatelet effect at 24 hours versus 1 hour for aggregation responses as measured by collagen induced platelet aggregation measured by Multiplate®. This is unknown and has never been demonstrated with any aspirin formulation.

TABLE 3
Change in Platelet Aggregation after
Administration of 325 mg ER ASA

| | | | | |
|---|---|---|---|---|
| 325 mg ER ASA | | | | |
| (post dose) | 1 h | 12 h | 16 h | 24 h |

LTA (Max %)

| | | | | |
|---|---|---|---|---|
| 1 mM Arachidonic Acid | 3 ± 2 | 4 ± 1 | 3 ± 1 | 3 ± 1 |
| 2 mM Arachidonic Acid | 5 ± 2 | 5 ± 1 | 5 ± 2 | 5 ± 1 |
| 2 ug/ml Collagen | 46 ± 20 | 36 ± 20 | 34 ± 23 | 47 ± 18 |
| 4 ug/ml Collagen | 51 ± 22 | 43 ± 21 | 47 ± 21 | 58 ± 20 |

Multiplate ® (AU*Min)

| | | | | |
|---|---|---|---|---|
| Arachidonic Acid | 16 ± 8 | 14 ± 5 | 19 ± 14 | 18 ± 9 |
| Collagen | 32 ± 10 | 34 ± 13 | 40 ± 21 | 43 ± 12* |

*indicates statistical significance

The results for 162.5 mg ER ASA (Durlaza®) and 325 mg ER ASA are in stark contrast to 75 mg IR ASA. As explained in the Background of the Invention section above, Würtz et al. shows a significant increase in both AA induced and collagen induced platelet aggregation 24 hours after administration of 75 mg IR ASA. Whereas, for 162.5 mg ER ASA (Durlaza®) and 325 mg ER ASA, surprisingly and unexpectedly, there is no significant increase in platelet aggregation after either AA induced or collagen induced platelet aggregation 24 hours after administration. In fact, for 325 mg ER ASA there is a significant decrease in collagen-induced platelet aggregation at 24 hours after administration representing a dose sensitive response. See Table 3 and FIG. 4.

B. Changes in ARU

### Method

The VerifyNow® is a turbidimetric based optical detection system that measures platelet aggregation in the whole blood and reports the optical signals as aspirin reaction units (“ARU”). The percent change in ARU over 24 hours (“ARU₂₄”) is calculated from 1 hour post dose using the following formulas: ARU₁₂(%)=((ARU₁−ARU₁₂)/ARU₁)*100; ARU₁₆(%)=((ARU₁−ARU₁₆)/ARU₁)*100 and ARU₂₄(%)=((ARU₁−ARU₂₄)/ARU₁)*100.

### Results

On average, there were no statistically significant changes from the 1 hour post dose in either 162.5 mg ER ASA (Durlaza®) dose or 325 mg ER ASA dose (Table 4, 162.5 mg and Table 5, 325 mg). At 16 hours post 325 mg ER ASA dose there was a significant increase in ARU (p=0.0313 using non-parametric test) compared to 1 hour, however statistical significance was found only for the median ARU and not the mean ARU.

TABLE 4
Changes in ARU at Visit 3

| ARU (%) | | | | |
|---|---|---|---|---|
| Timepoint | Statistic | Raw | Change | p-Value |
| 1 hour | n | 37 | — | — |
| | Mean (SD) | 485(69) | — | — |
| | Median | 478 | — | — |
| | Range | 369-594 | — | — |
| 12 hours | n | 39 | 37 | — |
| | Mean (SD) | 504(69) | −6(20) | 0.0998* |
| | Median | 502 | −1 | 0.2150^{#} |
| | Range | 367-591 | −52-29 | — |
| 16 hours | n | 39 | 37 | — |
| | Mean (SD) | 503(64) | −5(17) | 0.0653* |
| | Median | 487 | −5 | 0.0727^{#} |
| | Range | 390-599 | −47-28 | — |
| 24 hours | n | 39 | 36 | — |
| | Mean (SD) | 488(72) | −2(20) | 0.5574* |
| | Median | 471 | 0 | 0.7002^{#} |
| | Range | 384-592 | −48-31 | — |

TABLE 5
Changes in ARU at Visit 6

| ARU (%) | | | | |
|---|---|---|---|---|
| Timepoint | Statistic | Raw | Change | p-Value |
| 1 hour | n | 9 | — | — |
| | Mean (SD) | 470(85) | — | — |
| | Median | 451 | — | — |
| | Range | 350-589 | — | — |
| 12 hours | n | 7 | 7 | — |
| | Mean (SD) | 481(59) | −1(11) | 0.8968* |
| | Median | 498 | 2 | 1.0000^{#} |
| | Range | 406-576 | −18-10 | — |
| 16 hours | n | 7 | 7 | — |
| | Mean (SD) | 521(77) | −9(18) | 0.2351* |
| | Median | 569 | −1 | 0.0313^{#} |
| | Range | 407-591 | −50-0 | — |
| 24 hours | n | 7 | 7 | — |
| | Mean (SD) | 506(60) | −6(14) | 0.2947* |
| | Median | 504 | −2 | 0.2969^{#} |
| | Range | 408-577 | −27-16 | — |

C. PlateletMapping® Assay

### Method

The Plateletmapping® assay (Plateletmapping is a registered trademark of Haemoscope Corp.) was conducted using a TEG®-6S analyzer (TEG is a registered trademark of Haemoscope Corp.) and results included parameters: (1) clot strength at maximum amplitude (“MA”), (2) reaction time to end of thrombin burst (“R”), (3) fibrin stimulated maximum amplitude (“MA fibrin”), (4) adenosine diphosphate stimulated maximum amplitude (“MA ADP”), (5) adenosine diphosphate stimulated aggregation (“ADP Agg”), (6) arachidonic acid stimulated MA (“MA AA”), and (7) arachidonic acid stimulated aggregation (“AA Agg”). For summary purposes the change in each parameter over 24 hours is calculated by subtracting the result at 24 hours from the result at 1 hour (i.e., absolute difference).

### Results

Overall, the Plateletmapping® assay indicated that both 162.5 mg ER ASA (Durlaza®) and 325 mg ER ASA maintained platelet aggregation inhibition over a 24 hour period. See Tables 6 and 7.

TABLE 6
Change in Platetletmapping ® Assay Parameters at Visit 3

| Param- | | | Raw | | % Change | | |
|---|---|---|---|---|---|---|---|
| eter | Timepoint | n | Mean (SD) | | Mean (SD) | | p-Value |
| MA | 1 hour | 36 | 65 | (5) | — | | — |
| (mm) | 12 hours | 39 | 65 | (5) | −0 | (3) | 0.5952*; 0.6663^{#} |
| | 16 hours | 38 | 66 | (5) | −1 | (3) | 0.0384*; 0.0425^{#} |
| | 24 hours | 39 | 65 | (5) | −1 | (3) | 0.2746*; 0.1806^{#} |
| R | 1 hour | 36 | 6 | (2) | — | | — |
| (min) | 12 hours | 39 | 6 | (2) | 0 | (2) | 0.7433*; 0.6300^{#} |
| | 16 hours | 38 | 6 | (2) | 1 | (2) | 0.0811*; 0.0740^{#} |
| | 24 hours | 39 | 5 | (2) | 1 | (2) | 0.0225*; 0.0318^{#} |
| MA | 1 hour | 34 | 21 | (11) | — | | — |
| fibrin | 12 hours | 38 | 22 | (13) | −1 | (12) | 0.5090*; 0.1556^{#} |
| (mm) | 16 hours | 37 | 23 | (12) | −2 | (8) | 0.1857*; 0.3531^{#} |
| | 24 hours | 39 | 21 | (12) | −1 | (9) | 0.6756*; 0.5335^{#} |
| MA | 1 hour | 34 | 53 | (15) | — | | — |
| ADP | 12 hours | 38 | 53 | (16) | 2 | (15) | 0.5313*; 0.6557^{#} |
| (mm) | 16 hours | 34 | 57 | (13) | −1 | (10) | 0.6932*; 0.5395^{#} |
| | 24 hours | 39 | 53 | (17) | 0 | (14) | 0.8509*; 0.4043^{#} |
| MA AA | 1 hour | 34 | 39 | (17) | | | |
| (mm) | 12 hours | 38 | 36 | (19) | 3 | (13) | 0.1388*; 0.1966^{#} |
| | 16 hours | 36 | 38 | (18) | 2 | (14) | 0.4953*; 0.6020^{#} |
| | 24 hours | 39 | 42 | (16) | −3 | (15) | 0.3306*; 0.4999^{#} |
| AA | 1 hour | 34 | 42 | (37) | — | | — |
| Agg | 12 hours | 38 | 39 | (38) | 3 | (29) | 0.5667*; 0.3522^{#} |
| (%) | 16 hours | 36 | 36 | (36) | 7 | (22) | 0.0917*; 0.0243^{#} |
| | 24 hours | 39 | 47 | (35) | −3 | (31) | 0.5396*; 0.8036^{#} |

TABLE 7
Changes in Plateletmapping ® Assay Parameters at Visit 6

| Param- | | | Raw | | % Change | | |
|---|---|---|---|---|---|---|---|
| eter | Timepoint | n | Mean (SD) | | Mean (SD) | | p-Value |
| MA | 1 hour | 9 | 65 | (4) | — | | — |
| (mm) | 12 hours | 10 | 65 | (4) | −1 | (3) | 0.5902*; 0.5898^{#} |
| | 16 hours | 10 | 65 | (3) | −1 | (2) | 0.3401*; 0.3008^{#} |
| | 24 hours | 10 | 65 | (3) | 0 | (3) | 0.6058*; 0.5703^{#} |
| R | 1 hour | 9 | 6 | (2) | — | | — |
| (min) | 12 hours | 10 | 5 | (2) | 1 | (2) | 0.4195*; 0.2891^{#} |
| | 16 hours | 10 | 5 | (2) | 0 | (2) | 0.6855*; 0.5547^{#} |
| | 24 hours | 10 | 5 | (2) | 0 | (2) | 0.6021*; 0.8906^{#} |
| MA | 1 hour | 9 | 21 | (13) | — | | — |
| fibrin | 12 hours | 10 | 18 | (10) | 3 | (12) | 0.4576*; 0.6719^{#} |
| (mm) | 16 hours | 10 | 18 | (10) | 3 | (12) | 0.4327*; 0.4258^{#} |
| | 24 hours | 10 | 20 | (11) | 2 | (15) | 0.7649*; 0.5469^{#} |
| MA | 1 hour | 9 | 56 | (13) | — | | — |
| ADP | 12 hours | 10 | 59 | (10) | −3 | (11) | 0.3983*; 0.4961^{#} |
| (mm) | 16 hours | 10 | 56 | (14) | −0 | (13) | 0.9960*; 0.9102^{#} |
| | 24 hours | 10 | 59 | (10) | −4 | (11) | 0.2915*; 0.7344^{#} |
| MA AA | 1 hour | 9 | 41 | (16) | — | | — |
| (mm) | 12 hours | 10 | 43 | (15) | −3 | (14) | 0.5076*; 0.5703^{#} |
| | 16 hours | 10 | 44 | (13) | −4 | (17) | 0.5203*; 0.5703^{#} |
| | 24 hours | 10 | 48 | (14) | −8 | (18) | 0.1997*; 0.4258^{#} |
| AA | 1 hour | 9 | 45 | (36) | — | | — |
| Agg | 12 hours | 10 | 53 | (30) | −13 | (26) | 0.1811*; 0.2500^{#} |
| (%) | 16 hours | 10 | 55 | (24) | −13 | (37) | 0.3317*; 0.3008^{#} |
| | 24 hours | 10 | 61 | (34) | −21 | (39) | 0.1449*; 0.4609^{#} |

D. Comparison of Platelet Function and Aggregation at Trough Levels

### Method

The platelet function and aggregation measures at trough level were compared between the 81 mg immediate release ASA, 162.5 mg ER ASA (Durlaza®) and 325 mg ER ASA groups. For this study the trough level data are from the following timepoints: (1) for 81 mg—the data at Visit 2; (2) for 162.5 mg ER ASA (Durlaza®)—the data at Visit 3 pre-dose; and (3) for 325 mg ER ASA—the data at Visit 6 pre-dose.

### Results

No significant changes were observed in platelet function measures evaluated by PlateletWorks® (PlateletWorks is a registered trademark of Helena Laboratories Corp.) at the trough level. See Table 8. This is unknown for patients with Diabetes and CVD or multiple risk factors. For platelet aggregation measures it was observed that the 2 μg/mL collagen-induced platelet aggregation was significantly higher at the trough level from subjects dosed previously with 162.5 mg ER ASA (Durlaza®; p=0.002 (Max) and 0.009 (Final)). No significant changes were observed in collagen- and ADP-induced platelet aggregation at the trough level between 162.5 mg ER ASA (Durlaza®) and 81 mg ASA-IR dosing. See Table 8. No significant changes were observed in platelet function measures evaluated by PlateletWorks® and platelet aggregation measures at the trough level between 162.5 mg ER ASA (Durlaza®), 325 mg ER ASA and 81 mg ASA-IR dosing. See Table 8.

TABLE 8
Platelet Function and Aggregation at Trough Levels

| | Visit 2 vs. Visit 3 pre dose | | | | | |
|---|---|---|---|---|---|---|
| | | 162.5 mg | | Visit 2 vs. Visit 6 pre dose | | |
| LTA (%) | 81 mg IR | (Durlaza ®) | p-value | 81 mg IR | 325 mg ER | p-value |
| 1 mM AA (Max) | 5 ± 12 | 5 ± 9 | 1 | 4 ± 2 | 3 ± 2 | 0.27 |
| 2 mM AA (Final) | 5 ± 12 | 4 ± 8 | 0.66 | 3 ± 2 | 3 ± 2 | 1 |
| 2 mM AA (Max) | 7 ± 14 | 5 ± 3 | 0.38 | 4 ± 2 | 4 ± 2 | 1 |
| 2 mM AA (Final) | 6 ± 13 | 5 ± 4 | 0.64 | 3 ± 2 | 4 ± 3 | 0.27 |
| 2 μg/mL Collagen (Max) | 57 ± 24 | 50 ± 24 | 0.19 | 68 ± 11 | 47 ± 15 | 0.002 |
| 2 μg/mL Collagen (Final) | 54 ± 25 | 49 ± 24 | 0.36 | 67 ± 12 | 48 ± 17 | 0.009 |
| 4 μg/mL Collagen (Max) | 66 ± 21 | 59 ± 23 | 0.15 | 73 ± 12 | 59 ± 19 | 0.06 |
| 4 μg/mL Collagen (Final) | 65 ± 20 | 57 ± 21 | 0.08 | 71 ± 13 | 56 ± 21 | 0.07 |
| 5 μM ADP (Max) | 59 ± 14 | 55 ± 13 | 0.18 | 61 ± 10 | 58 ± 14 | 0.58 |
| 5 μM ADP (Final) | 50 ± 17 | 48 ± 15 | 0.58 | 52 ± 14 | 49 ± 15 | 0.65 |
| VerifyNow ® Aspirin | 477 ± 72 | 511 ± 65 | 0.03 | 475 ± 102 | 484 ± 79 | 0.83 |
| Assay (ARU) | | | | | | |

MultiPlate ® (AU)

| | | | | | | |
|---|---|---|---|---|---|---|
| ADP | 70 ± 24 | 79 ± 30 | 0.14 | 77 ± 15 | 68 ± 28 | 0.38 |
| AA | 17 ± 14 | 18 ± 13 | 0.74 | 24 ± 13 | 17 ± 10 | 0.19 |
| Collagen | 36 ± 17 | 36 ± 16 | 1 | 47 ± 18 | 39 ± 14 | 0.28 |

PlateletWorks ® (% Agg)

| | | | | | | |
|---|---|---|---|---|---|---|
| ADP | 83 ± 15 | 84 ± 14 | 0.76 | 84 ± 10 | 85 ± 4 | 0.38 |
| AA | 44 ± 19 | 43 ± 15 | 0.79 | 44 ± 16 | 32 ± 11 | 0.66 |
| Collagen | 47 ± 17 | 49 ± 15 | 0.57 | 44 ± 12 | 30 ± 17 | 0.04 |

TEG PlateletMapping ®

| | | | | | | |
|---|---|---|---|---|---|---|
| MA (clot strength, mm) | 65 ± 5 | 65 ± 5 | 1 | 64 ± 3 | 64 ± 3 | 1 |
| R (Clotting time, min) | 6.8 ± 1.5 | 6.4 ± 1.8 | 0.28 | 7.1 ± 0.7 | 6.7 ± 1.9 | 0.54 |
| MA-fibrin (mm) | 17 ± 11 | 19 ± 10 | 0.4 | 24 ± 12 | 18 ± 10 | 0.24 |
| MA-ADP (mm) | 50 ± 18 | 55 ± 16 | 0.19 | 52 ± 11 | 56 ± 9 | 0.39 |
| MA-AA (mm) | 39 ± 15 | 43 ± 16 | 0.25 | 54 ± 9 | 45 ± 13 | 0.08 |
| ADP Aggregation | 74 ± 25 | 74 ± 25 | 1 | 73 ± 21 | 76 ± 23 | 0.76 |
| AA Aggregation | 48 ± 29 | 53 ± 34 | 0.48 | 75 ± 17 | 60 ± 23 | 0.11 |

E. Correlation of Platelet Turnover Measures with Platelet Function Measures

### Method

The Platelet turnover measures included Mean Platelet Volume (“MPV”), Immature Platelet Fraction (“IPF”) and thrombopoietin level. The Platelet function measures included platelet count, ADP aggregation, AA aggregation and collagen aggregation.

### Results

Significant correlation was observed at Visit 3 between: (1) platelet count and MPV (inversely correlated, p-value=0.0006); (2) platelet count and IPF (inversely correlated, p-value=0.0021); and ADP aggregation and MPV (inversely correlated, p-value=0.0423). See Table 9. Additionally, there is a positive correlation between MPV and IPF. These results indicate high platelet turnover.

TABLE 9
Correlation of Platelet Turnover Measures
with Platelet Function Measures

| | | | |
|---|---|---|---|
| Platelet Function | | Mean Platelet | Immature |
| Measure | Statistics | Volume | Platelet Fraction |

Visit 2

| | | | |
|---|---|---|---|
| Platelet Count | Correlation | −0.3193* | −0.2113* |
| | p-Value | 0.0854 | 0.2624 |
| | n | 30 | 30 |
| ADP Aggregation | Correlation | −0.0594* | −0.1952* |
| | p-Value | 0.7552 | 0.3012 |
| | n | 30 | 30 |
| AA Aggregation | Correlation | −0.1547* | −0.2328* |
| | p-Value | 0.4409 | 0.2426 |
| | n | 27 | 27 |
| Collagen Aggregation | Correlation | 0.0512* | −0.1986* |
| | p-Value | 0.788 | 0.2928 |
| | n | 30 | 30 |

Visit 3

| | | | |
|---|---|---|---|
| Platelet Count | Correlation | −0.5614* | −0.5166* |
| | p-Value | 0.0006 | 0.0021 |
| | n | 34 | 33 |
| ADP Aggregation | Correlation | −0.3611* | −0.3210* |
| | p-Value | 0.0423 | 0.0783 |
| | n | 32 | 31 |
| AA Aggregation | Correlation | −0.2395* | −0.3297* |
| | p-Value | 0.1794 | 0.0653 |
| | n | 33 | 32 |
| Collagen Aggregation | Correlation | −0.1270* | −0.1547* |
| | p-Value | 0.496 | 0.4142 |
| | n | 31 | 30 |

### II. Changes in TxB2

A. Changes in Serum TxB2 Levels

### Method

Blood samples were collected in unanticoagulated glass tubes (Becton-Dickinson, Franklin Lakes, N.J.) and allowed to clot for 30 minutes in a 37° C. water bath. Clotted tubes were then centrifuged for 15 minutes at 3000 g at 4° C. and serum was stored at −70° C. until analysis. Serum TxB2 was measured by ELISA (Cayman Chemical Company, Ann Arbor, Mich.).

### Results

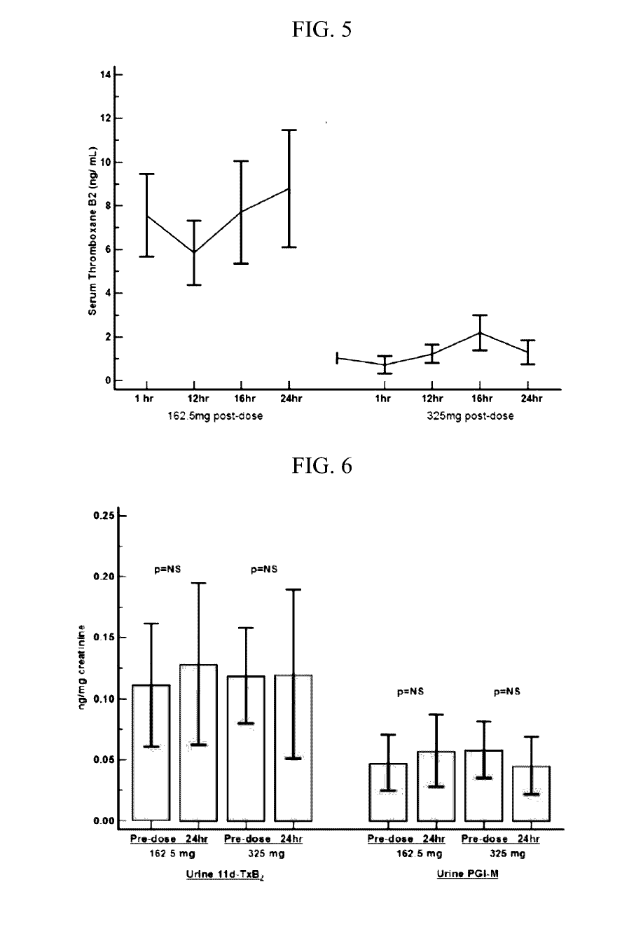

During 162.5 mg ER ASA (Durlaza®) daily administration, there was a surprising trend towards lower serum TxB2 levels observed at 12 hrs post-dose compared to 1 hr post-dose (8.1±12.3 vs. 5.8±8.6, p=0.09). See FIG. 5. Mean 1-24 hr serum TxB2 levels were lower with 325 mg daily ER ASA compared to 162.5 mg daily ER ASA (Durlaza®; p=7.3±10.7 ng/ml vs. 1.5±1.9 ng/ml, p=0.002) See FIG. 5. This result is in contrast to Würtz et al. that shows a significant increase in serum TxB2 levels 24 hours after administration of 75 mg IR ASA. Further, 325 mg ER ASA lowered serum TxB2 levels further than 162.5 mg ER ASA (Durlaza®) indicating a dose sensitive response.

B. Changes in Urinary TxB2 and PGI₂-M

### Method

Urine samples were stored at −70° C. were stored and shipped on dry ice to the Eicosanoid Core Laboratory at Vanderbilt University (Nashville, Tenn.) for analysis. Urinary 11-dh TxB2 and prostacyclin metabolite (PGI₂-M) were measured using gas chromatography-mass spectrometry (GC/MS). The precision of the assay is +/−7% and accuracy is 90%. Urinary creatinine levels were measured using a test kit from Enzo Life Sciences (Farmingdale, N.Y., USA). The urinary 11-dh TxB2 and PGI2-M levels in each sample were normalized using the urinary creatinine level of the sample and expressed in ng/mg creatinine.

### Results

No significant changes were observed in urinary metabolites (TxBM2 and PGI₂-M) following administration of either 162.5 mg ER ASA (Durlaza®) or 325 mg ER ASA at 24 hours post-dose versus pre-dose. See FIG. 6. This indicates that the majority of ASA from the ER ASA compositions is deacetylated in the liver and does not survive intact through the first pass.

### III. Anti-Inflammatory Properties

A. Mean Reactive Hyperemia Index (RHI)

### Method

Endothelial function was assessed using the ENDOPAT system (Itamar Medical, Israel), a pulsatile arterial tonometry-based diagnostic device, at Visit 2, Visit 3, Visit 5, and Visit 6 predose. The reactive hyperemia index (“RHI”), a measure of endothelial function, and the augmentation index (“AI”), a measure of arterial stiffness, were determined. Endothelial dysfunction is defined as RHI<1.67. Normal arterial stiffness is defined as an AI −30% to −10%, increased arterial stiffness as an AI>−10% to 10%, and abnormal arterial stiffness as an AI>10%. The RHI measures vessel pliability which is a surrogate for nitric oxide production. The production of nitric oxide inhibits platelet aggregation. Thus, determining nitric oxide production following Durlaza® dosing is important to understanding its affect on platelet inhibition.

### Results

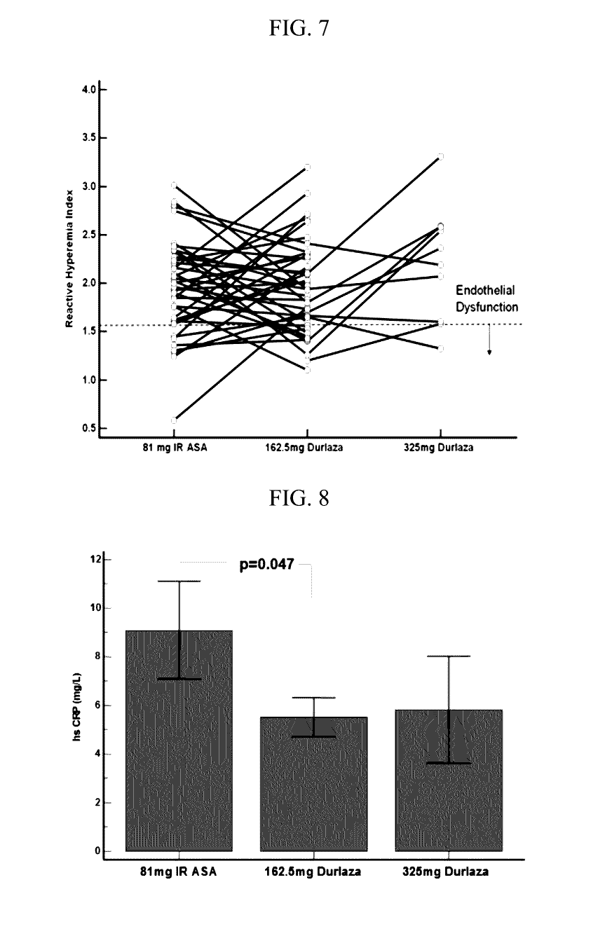

Increased or abnormal arterial stiffness was observed in 68% (27/40) of the total study population as measured by AI. There was no difference in RHI between 81 mg IR aspirin and 162.5 mg ER ASA (Durlaza®; 1.9±0.5 for both). See Table 10 and FIG. 7. Surprisingly, in patients with HPT, treatment with 325 mg ER ASA was associated with an improvement in RHI as compared to 81 mg IR aspirin (2.3±0.6 vs. 1.7±0.5, p=0.034). See Table 10 and FIG. 7. RHI index was statistically significantly different between 162.5 mg ER ASA dose (Durlaza®) and 325 mg dose (2.3±0.6 vs. 1.9±0.5, p=0.0028). See FIG. 7.

TABLE 10
Reactive Hyperemia Index

| | | | | | |
|---|---|---|---|---|---|
| | | | | | RHI < 1.67 |
| N | Mean | SD | Minimum | Maximum | (n, %) |

Visit 2

| | | | | | | | |
|---|---|---|---|---|---|---|---|
| (81 mg IR ASA) | 39 | 1.94 | 0.49 | 0.58 | 3.01 | 111 | (27.5) |

Visit 3

| | | | | | | | |
|---|---|---|---|---|---|---|---|
| (162.5 mg ER ASA; | 40 | 1.95 | 0.48 | 1.1 | 3.2 | 9 | (20) |
| Durlaza ®) | | | | | | | |

HPT Group
Visit 5

| | | | | | | | |
|---|---|---|---|---|---|---|---|
| (81 mg IR ASA) | 9 | 1.74 | 0.45 | 1.23 | 2.27 | 4 | (44) |

Visit 6

| | | | | | | | |
|---|---|---|---|---|---|---|---|
| (325 mg ER ASA) | 9 | 2.3 | 0.59 | 1.32 | 3.31 | 2 | (22) |

Visit 2 vs. Visit 3 (p=0.92)

Visit 2 vs. Visit 5 (p=0.25)

Visit 5 vs. Visit 6 (p=0.034)

B. High Sensitive C-Reactive Protein (hs-CRP) Test

### Method

High Sensitive C-Reactive Protein (“hs-CRP”) was measured using a Seimens Immulite 2000® Xpi analyzer (Immulite 2000 is a registered trademark of Siemens Healthcare Diagnostic, Inc.), prior to 162.5 mg ER ASA (Durlaza®) dosing at Visit 2 pre-dose, at Visit 3 pre-dose and Visit 3 at 24-hr post-dose for the 162.5 mg ER ASA (Durlaza®) dose, and at Visit 6 pre-dose and Visit 6 at 24-hr post-dose for the 325 mg ER ASA dose.

### Results

The frequency of elevated CRP was very high in the total study population with 47% of patients meeting the criteria for elevated CRP during 81 mg IR ASA therapy. CRP levels decreased with 162.5 mg ER ASA (Durlaza®) therapy as compared to 81 mg IR aspirin and remained lower with 325 mg ER ASA See Table 11 and FIG. 8. There was no difference between CRP level between pre- and 24 hour post-measurements with 162.5 mg ER ASA (Durlaza®) and 325 mg ER ASA. See Table 18 and FIG. 8.

TABLE 11
Mean High Sensitive C-Reactive Protein (hs-CRP)

| | | | | | |
|---|---|---|---|---|---|
| | | | | | hs-CRP > |
| | | | | | 3 mg/L |
| N | Mean | SD | Minimum | Maximum | (n, %) |

V2 pre-dose

| | | | | | | | |
|---|---|---|---|---|---|---|---|
| (81 mg IR ASA) | 40 | 9.62 | 13.2 | 0.44 | 57.4 | 23 | (57) |

V3 pre-dose

| | | | | | | | |
|---|---|---|---|---|---|---|---|
| (162.5 mg ER ASA | 39 | 5.52 | 4.98 | 0.6 | 16.7 | 16 | (41) |
| (Durlaza ®)) | | | | | | | |

V3 24 hrs post dose

| | | | | | | | |
|---|---|---|---|---|---|---|---|
| (162.5 mg ER ASA | 37 | 5.59 | 5.14 | 0.53 | 17.9 | 13 | (37) |
| (Durlaza ®)) | | | | | | | |

V6 pre-dose

| | | | | | | | |
|---|---|---|---|---|---|---|---|
| (325 mg ER ASA) | 10 | 5.82 | 6.91 | 0.6 | 19 | 4 | (40) |

V6 24 hrs post dose

| | | | | | | | |
|---|---|---|---|---|---|---|---|
| (325 mg ER ASA) | 10 | 4.19 | 5.12 | 0.52 | 15.8 | 4 | (40) |

V2_{predose} vs. V3_{predose} (p=0.047)

V2_{predose} vs. V3_{postdose} (p=0.95)

V2_{predose} VS. V6_{postdose} (p=0.50)

### Example 3—Comparison of IR ASA and ER ASA on Excretion of Thromboxane and Prostacyclin Metabolites

In a separate study, urinary TxB2 and prostacyclin metabolite levels were reduced after administration of ER ASA compositions as compared to a placebo. For urinary TxB2 the reduction was dose dependent as 162.5 mg ER ASA (Durlaza®) significantly reduced TxB2 levels as compared to 81 mg ER ASA. Finally, for all ER ASA doses urinary TxB2 is reduced significantly more than prostacyclin metabolites.

Methods

Subjects of Group 1 were randomly administered a placebo, 81 mg IR ASA and 162.5 mg IR ASA. Subjects of Group 2 were randomly administered a placebo, 81 mg ER ASA and 162.5 mg ER ASA (Durlaza®). Each subject was prohibited from taking any aspirin for two weeks prior to being administered the randomly chosen drug daily for 1 week followed by a urine collection 24 hours after dosing. This protocol was repeated two additional times. For each urine collection TxB2 and 2, 3-dinor-6-keto-PGF1α (“prostacyclin”) levels were measured using gas chromatography-mass spectrometry (“GC/MS”). Statistical analysis was done using Wilcoxon rank sum test for continuous variables and the Pearson test for categorical variables.

### Results

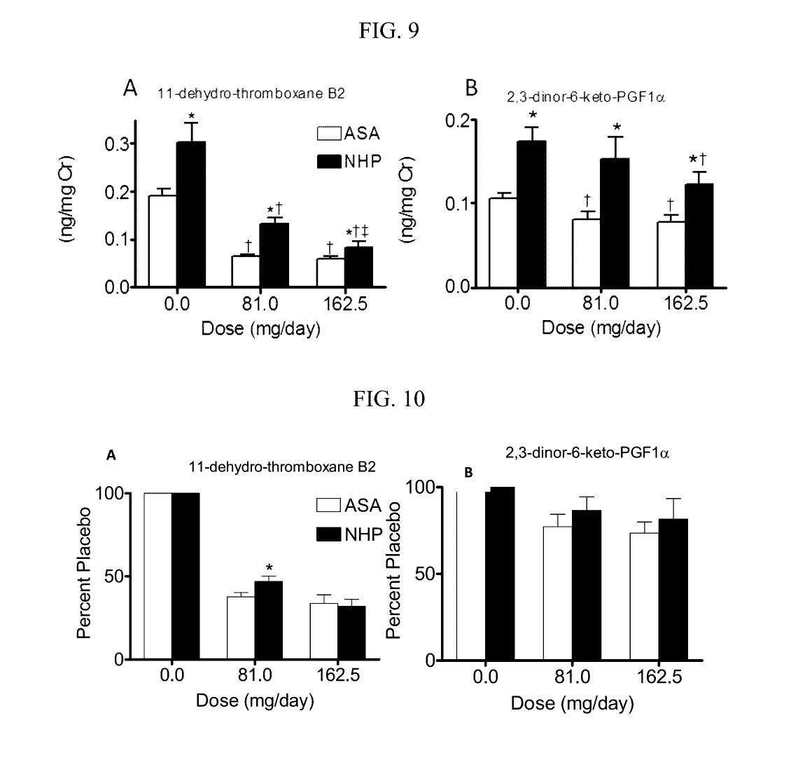

Basal urinary TxB2 levels 24 hours post-dosing were significantly reduced over placebo for each of 81 mg IR ASA, 162.5 mg IR ASA, 81 mg ER ASA and 162.5 mg ER ASA (Durlaza®). See FIG. 9, panel A. Further, TxB2 levels in subjects administered 162.5 mg ER ASA (Durlaza®) were significantly lower than those administered 81 mg ER ASA. For ER ASA, there was a dose-dependent effect of TxB2 synthesis and only the higher dose reduced basal prostacyclin synthesis. See FIG. 9, panels A and B.

Basal prostacyclin levels were significantly reduced over placebo for each of 81 mg IR ASA, 162.5 mg IR ASA and 162.5 mg ER ASA (Durlaza®). See FIG. 9, panel B.

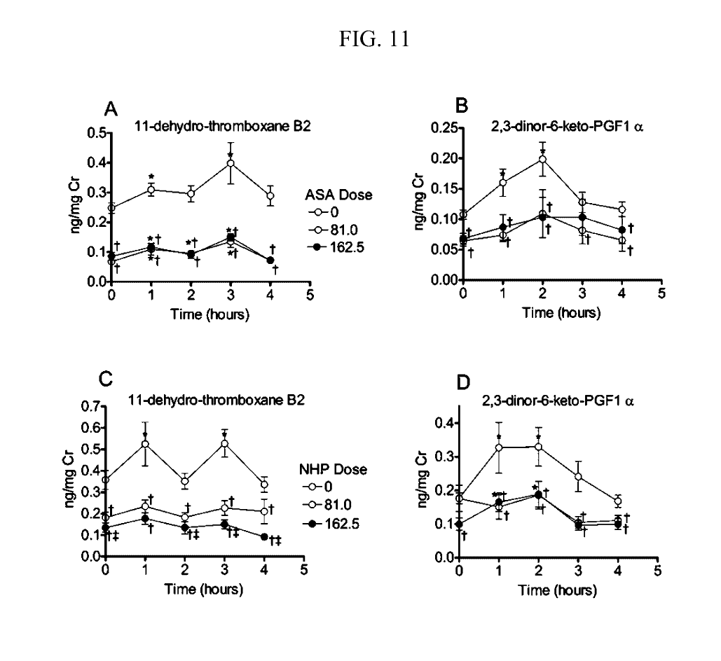

Despite significantly reducing levels of both TxB2 and prostacyclin immediate-release and extended-release aspirin selectively inhibit basal TxB2 production over prostacyclin production at doses of 81 and 162.5 mg/d. See FIG. 10.

Bradykinin-stimulated prostacyclin levels were significantly reduced over placebo for each of 81 mg IR ASA, 162.5 mg IR ASA and 162.5 mg ER ASA (Durlaza®). See FIG. 11, panels B and D.

Bradykinin-stimulated TxB2 levels 4 hours post-dosing were significantly reduced over placebo for each of 81 mg IR ASA, 162.5 mg IR ASA, 81 mg ER ASA and 162.5 mg ER ASA (Durlaza®). See FIG. 11, panels A and C. Further, TxB2 levels in subjects administered 162.5 mg ER ASA (Durlaza®) were significantly lower than those administered 81 mg ER ASA. See FIG. 11, panel C.

Unexpectedly, despite the fact that IR ASA and ER ASA both contain acetylsalicylic acid, there is a dose-dependent effect for ER ASA on TxB2 synthesis which is not seen for IR ASA. Additionally, only the higher dose of ER ASA significantly reduced prostacyclin levels whereas both 81 mg and 162.5 mg IR ASA significantly reduced prostacyclin levels.

### Example 4—Comparison of IR ASA and ER ASA on Pro-Inflammatory Interleukin Levels

ER ASA compositions surprisingly have either no effect or reduce pro-inflammatory interleukin levels as compared to IR ASA compositions which increase levels of these interleukins.

### Methods

Tests were setup and conducted similar to that in Example 3. Interleukin-8 (“IL-8”) serum levels (and Interleukin-6 levels, not shown) were measured both prior to and after administration of 0, 81 or 162.5 mg of either IR ASA or ER ASA.

### Results

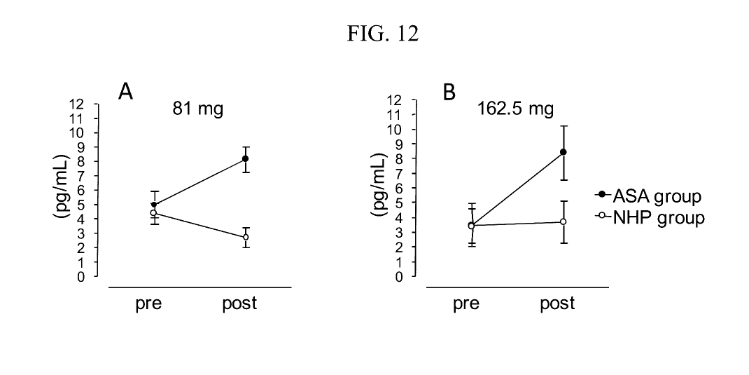

Bradykinin-stimulated IL-8 (and IL-6) levels were divergent when comparing IR ASA to ER ASA. See FIG. 12, panels A and B. Specifically, 81 mg ER ASA reduced IL-8 levels whereas 81 mg IR ASA increased IL-8 levels. See FIG. 12, panel A. For 162.5 mg, ER ASA (Durlaza®) had no significant effect on IL-8 levels whereas IR ASA again increased IL-8 levels. This is significant because IL-8 and IL-6 are pro-inflammatory cytokines that promote platelet production and aggregation. These results were unexpected as both IR ASA and ER ASA have the same active drug and 81 mg IR ASA is bioequivalent to 162.5 mg ER ASA (Durlaza®).

What is claimed is:

1. A method of inhibiting platelet aggregation comprising orally administering to a human in need thereof a controlled-release aspirin composition comprising acetylsalicylic acid (ASA) at an amount from about 81 milligrams to about 325 milligrams, wherein the method provides a level of platelet aggregation inhibition within about 1 hour of administration and wherein the level of platelet aggregation inhibition remains significantly unchanged from about 1 hour after administration to at least about 24 hours after administration.

2. The method of claim 1, wherein platelet aggregation is measured by a turbidimetric based optical detection system.

3. A method of reducing serum thromboxane B2 levels comprising orally administering to a human in need thereof a controlled-release aspirin composition comprising acetylsalicylic acid (ASA) at an amount from about 81 milligrams to about 325 milligrams, wherein the method provides a reduced serum thromboxane B2 level within 1 hour of administration and wherein the serum thromboxane level remains significantly unchanged from about 1 hour after administration to at least about 24 hours after administration.

4. The method of claim 3, wherein the administration of 325 milligrams ASA provides a significantly lower serum thromboxane B2 level as compared to administration of 162.5 milligrams ASA.

5. The method of claim 3, wherein the method further provides no significant change in urinary levels of thromboxane B2.

6. The method of claim 3, wherein the composition comprises 81 milligrams ASA and the method provides no significant change in urinary levels of 2,3-dinor-6-keto-PGF1α.

7. The method of claim 3 wherein the composition comprises 162.5 milligrams ASA and the method provides lower urinary thromboxane B2 levels than after administration of the controlled-release aspirin composition comprising about 81 milligrams ASA.

8. A method of reducing systemic or cardiovascular inflammation comprising orally administering to a human in need thereof a controlled-release aspirin comprising acetylsalicylic acid (ASA) at an amount from about 162.5 milligrams to about 325 milligrams, wherein the method provides at least one of:
a) stimulation of vascular production of nitric oxide as measured by a significant change in mean reactive hyperemia index score as measured by pulse amplitude tonometry compared to a mean reactive hyperemia index score measured by pulse amplitude tonometry prior to oral administration of the composition to the human;
b) a significantly reduced high sensitive C-reactive protein level compared to a high sensitive C-reactive protein level after oral administration to a human in need thereof of a bioequivalent amount of immediate-release aspirin; and
c) no significant change in interleukin-8 levels.

9. The method of claim 8, wherein the method provides a significant increase in mean reactive hyperemia index score as measured by pulse amplitude tonometry compared to a mean reactive hyperemia index score measured by pulse amplitude tonometry prior to oral administration of the composition to the human.

10. The method of claim 8, wherein the method provides a significantly reduced high sensitive C-reactive protein level compared to a high sensitive C-reactive protein level after oral administration to a human of a bioequivalent amount of immediate-release aspirin

11. The method of claim 8, wherein the method provides no significant change in interleukin-8 levels.

12. The method of claim 10, wherein the bioequivalent amount is 81 milligrams.

13. A method of preventing or treating cancer comprising orally administering to a human in need thereof a controlled-release aspirin composition comprising acetylsalicylic acid (ASA) at an amount from about 162.5 milligrams to about 325 milligrams.

14. The method of claim 13, wherein the method provides greater than 100 nanograms of ASA per milliliter of serum for at least 4 hours.

15. The method of claim 14, wherein the method provides greater than 30 nanograms of ASA per milliliter of serum for at least 8 hours.

16. The method of claim 15, wherein the method provides greater than 1000 nanograms of salicylic acid per milliliter of serum for at least 8 hours.

17. The method of claim 16, wherein the method provides greater than 200 nanograms of salicylic acid per milliliter of serum for at least 24 hours.

18. The method of claim 17 wherein the cancer is selected from the group consisting of breast cancer and colorectal cancer.

19. A method of treating or preventing a disease associated with increased thrombotic risk due to platelet activation, aggregation or production comprising orally administering to a human in need thereof a controlled-release aspirin composition comprising acetylsalicylic acid (ASA) at an amount from about 81 milligrams to about 325 milligrams, preferably from about 162.5 milligrams to 325 milligrams, wherein the method provides a level of platelet aggregation inhibition within about 1 hour of administration and wherein the level of platelet aggregation inhibition remains significantly unchanged from about 1 hour after administration to at least about 24 hours after administration.

20. The method of claim 19, wherein the disease is essential thrombocytosis or essential thrombocythemia.

21. A composition comprising an immediate release acetylsalicylic acid (IR) and an extended-release acetylsalicylic acid (ER) in a ratio from about 1:1 to about 6:1 IR:ER.

22. The composition of claim 21 wherein the IR:ER ratio is selected from the group consisting of 1:1, 1.5:1, 2:1, 2.5:1, 3:1, 3.5:1, 4:1, 4.5:1, 5:1, 5.5:1 and 6:1.
